(19) United States
(12) Patent Application Publication
(10) Pub. No.: US 2024/0398801 A1
(43) Pub. Date: Dec. 5, 2024

(54) TREATMENT OF LEUKEMIA BASED ON LEUKEMIA HIERARCHY IN A PATIENT

(71) Applicant: UNIVERSITY HEALTH NETWORK, Toronto (CA)

(72) Inventors: Andy G.X. ZENG, Toronto (CA); Jean G.X. WANG, Toronto (CA); John G.X. DICK, Toronto (CA)

(21) Appl. No.: 18/696,770

(22) PCT Filed: Sep. 30, 2022
(86) PCT No.: PCT/CA2022/051462

§ 371 Date: Mar. 28, 2024

Related U.S. Application Data
(60) Provisional application No. 63/251,597, filed on Oct. 2, 2021.

(51) Int. Cl. A61K 31/506; A61K 31/454; A61P 35/02; C12Q 1/6874; C12Q 1/6886

(52) U.S. Cl. CPC A61K 31/506; A61K 31/454; A61P 35/02; C12Q 1/6874; C12Q 1/6886; C12Q 2600/106; C12Q 2600/154; C12Q 2600/158; C12Q 2600/178

(57) ABSTRACT

There is described herein a method of predicting treatment response to a drug in a patient with leukemia, wherein the drug had been predetermined to preferentially target either primitive or mature leukemic cells, the method comprising: determining a primitiveness score using at least 3 genes in a test sample from the subject selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKRIC3, and GPR56.

### CROSS REFERENCE TO RELATED APPLICATIONS

This application claims priority to U.S. Provisional Patent Application No. 63/251,597 filed on Oct. 2, 2021, the contents of which are incorporated by reference in their entirety.

### FIELD OF THE INVENTION

The invention relates to methods and products for the treatment of leukemia patients, particularly acute myeloid leukemia (AML) patients. Specifically, the invention relates assessing a patient's leukemia hierarchy and selecting particular drugs based thereon.

### BACKGROUND

AML is a devastating disease characterized by extensive inter-patient and intra-patient heterogeneity. Poor outcomes are attributable to primary chemotherapy resistance and a high rate of relapse among patients who achieve remission, highlighting the inadequacy of standard chemotherapy as a means of curing AML for most patients. Recently a wide range of promising new therapies targeting diverse cellular mechanisms have been approved or are progressing through clinical trials, offering alternatives to chemotherapy. However, patient responses to these new therapies are heterogeneous and we lack a reliable way to select the best therapy for each patient.

Historically, two distinct approaches have evolved for understanding heterogeneity in AML and informing therapy selection: a genomic model and a stem-cell model. The discovery of the Philadelphia chromosome in 1960¹ sparked a series of cytogenetic studies that identified distinct cytogenetic drivers of AML. These cytogenetic groups, together with the French-American-British (FAB) morphological classification developed around the same time^{2,3}, led to a new disease classification system⁴. More recently, advances in genome sequencing have uncovered mutational drivers of AML and culminated in a refined genomic classification with important prognostic implications⁵. While this genomic model accounts for a major source of inter-patient heterogeneity, cells sharing the same driver mutation can exhibit functional differences⁶. Moreover, while some driver mutations can be directly targeted by inhibitors, genomic profiling is limited in its ability to predict benefit from therapies targeted to specific biological processes or signaling pathways.

The discovery of hematopoietic stem cells in 1961 and the development of quantitative assays to interrogate stem cell function⁷ motivated a parallel line of investigation into AML, wherein pioneering experiments revealed functional differences among blasts within individual patients in their cycling kinetics⁸⁻¹⁰, differentiation state¹¹⁻¹³ and self-renewal capacity¹⁴⁻¹⁶. Collectively, these studies conceptualized that AML is sustained by rare leukemia stem cells (LSCs)¹⁷, which were later formally identified through xenotransplantation studies¹⁸⁻²⁰. LSCs have since been shown to mediate relapse²¹, and LSC-based stemness scores have emerged as predictors of outcomes following chemotherapy²²⁻²⁸. However, as the stem cell model primarily captures intra-patient heterogeneity, it does not account for heterogeneity between patients beyond stemness properties. Furthermore, while LSCs are an important therapeutic target, this model offers limited guidance around therapy selection. Overall, these genomic and stem-cell models provide complementary insight into AML heterogeneity, yet neither model can overcome the therapy selection challenge independently. This highlights the need for a more comprehensive approach for understanding heterogeneity in AML and the means to better integrate the information derived from genomic and stem cell models.

Cancer has long been recognized as a caricature of normal tissue development^{29,30}. AML is one of the best studied cancer systems wherein malignant cells are organized into a hierarchy resembling normal blood development. Cellular hierarchies in AML can be distorted in different ways, depending on genetic alterations and cell of origin. For example, a strong differentiation block arising in a stem cell may result in a shallow, stem cell-dominant hierarchy. In other cases considerable-albeit aberrant-differentiation may occur resulting in a steep hierarchy wherein rare LSCs generate a bulk blast population with mature myeloid features. In this way, the composition of each patient's hierarchy likely reflects the functional impact of specific mutations on the disease-sustaining LSCs. Thus, interrogation of leukemic hierarchies provides an opportunity to potentially integrate features of the genetic and stem cell models of AML³¹. Single cell RNA-sequencing (scRNA-seq) has emerged as a powerful tool for dissecting cellular hierarchies^{32,33}, yet prohibitive costs restrict these studies to a limited number of patients. Without measuring these cellular hierarchies at scale in large clinical datasets, their relationship to therapy response remains unknown.

### SUMMARY OF THE INVENTION

In an aspect, there is provided a method of predicting treatment response to a drug in a patient with leukemia, wherein the drug had been predetermined to preferentially target either primitive or mature leukemic cells, the method comprising: determining the expression level of at least 3 genes in a test sample from the subject selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, and GPR56; calculating a primitiveness score comprising the weighted sum expression of each of the at least 3 genes; and either predicting that the patient will be sensitive to treatment by the drug if (i) the drug preferentially targets primitive leukemic cells and the calculated primitiveness score is high in reference to a control cohort of leukemia patients; or (ii) the drug preferentially targets mature leukemic cells and the calculated primitiveness score is low in reference to a control cohort of leukemia patients; or predicting that the patient will be resistant to treatment by the drug if (i) the drug preferentially targets primitive leukemic cells and the calculated primitiveness score is low in reference to a control cohort of leukemia patients; or (ii) the drug preferentially targets mature leukemic cells and the calculated primitiveness score is high in reference to a control cohort of leukemia patients.

In a preferred embodiment, the at least 3 genes consists of DNMT3B, LAPTM4B, CDK6, CPXM1, NGFRAP1, CD34, and GPR56.

In an aspect, there is provided a composition comprising a plurality of isolated nucleic acid sequences, wherein each isolated nucleic acid sequence hybridizes to: (a) the mRNA of at least 3 genes selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, GPR56; and/or (b) a nucleic acid complementary to a), wherein the composition is used to measure the level of expression of the at least 3 genes. In some embodiments, the at least 3 genes consists of DNMT3B, LAPTM4B, CDK6, CPXM1, NGFRAP1, CD34, and GPR56.

In an aspect, there is provided an array comprising, for each of at least 3 genes selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, GPR56, one or more polynucleotide probes complementary and hybridizable thereto. In some embodiments, the at least 3 genes consists of DNMT3B, LAPTM4B, CDK6, CPXM1, NGFRAP1, CD34, and GPR56

In an aspect, there is provided a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the method described herein.

In an aspect, there is provided a computer implemented product for predicting treatment response to a drug in a patient with leukemia, wherein the drug had been predetermined to preferentially target either primitive or mature leukemic cells, the computer implemented product comprising: (a) a means for receiving values corresponding to a subject expression profile comprising at least 3 genes from the subject selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, and GPR56; (b) a database comprising a reference expression profile representing a control, wherein the subject expression profile and the reference profile each have at least one value representing the expression level of at least 3 genes selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, GPR56; (c) a means for calculating a primitiveness score comprising the weighted sum expression of each of the at least 3 genes; and either (d) a means for outputting a prediction that that the patient will be sensitive to treatment by the drug if (i) the drug preferentially targets primitive leukemic cells and the calculated primitiveness score is high in reference to a control cohort of leukemia patients; or (ii) the drug preferentially targets mature leukemic cells and the calculated primitiveness score is low in reference to a control cohort of leukemia patients; or (e) a means for outputting a prediction that the patient will be resistant to treatment by the drug if (i) the drug preferentially targets primitive leukemic cells and the calculated primitiveness score is low in reference to a control cohort of leukemia patients; or (ii) the drug preferentially targets mature leukemic cells and the calculated primitiveness score is high in reference to a control cohort of leukemia patients.

In an aspect, there is provided a method for selecting a drug in a patient with leukemia, wherein the drug had been predetermined to preferentially target either primitive or mature leukemic cells, the method comprising the method of any one of claims 1-7, and further comprising selecting the drug if the patient had been predicted to be sensitive to treatment by the drug according to the patient's primitiveness score.

In an aspect, there is provided a drug for use in the treatment of leukemia in a patient, wherein the patient had been determined to be sensitive to the drug by the method described herein.

In an aspect, there is provided a use of a drug in the preparation of a medicament for the treatment of leukemia in a patient, wherein the patient is determined to be sensitive to the drug by the method described herein.

### BRIEF DESCRIPTION OF THE FIGURES

FIG. 1. Heterogeneous LSPC populations from single-cell RNA-seq. A) Diffusion map of re-annotated LSPC populations using feature weights from Self-Assembling Manifolds (SAM). B) Transcription Factor regulon activity in each malignant cell type, inferred through PySCENIC. C) Schematic of deconvolution approach using reference signatures from single cell RNA-seq populations. D) Enrichment of malignant cell types across sorted AML fractions, functionally defined as LSC+ (engrafting) or LSC− (non-engrafting) through xenotransplantation. E) Enrichment of malignant cell types in fractions sorted with different LSC markers. Log-pvalue of cell-type enrichment in specified fractions is depicted, with significant changes (p<0.05) marked with an asterisk.

FIG. 2. Capturing the cellular phenotypes of key genetic alterations. A) Principal component analysis of 812 diagnostic AML patients from TCGA, BEAT-AML, and Leucegene based on the composition of their cellular hierarchy. B) Relative abundance of each malignant cell type in each of the 812 patients. Each bar represents an individual patient and the distribution of colors throughout each bar represents the distribution of malignant cell populations within their leukemic hierarchy. C) Illustrations of the cellular organization of Primitive, GMP, and Mature hierarchies. D) Density plots depicting mutation combinations along the Primitive vs Mature axis (PC2). Mutation combinations are coloured by prognostic significance from Papaemmanuil et al (NEJM 2016), wherein red indicates adverse prognosis while green indicates favourable prognosis. E) Cytogenetic groups along the Primitive vs GMP axis (PC1). Cytogenetic alterations are coloured by prognostic significance. F) Survival outcomes of hierarchy clusters in TCGA.

FIG. 3. Shift towards primitive hierarchies at chemotherapy relapse. A) Paired diagnosis and relapse samples projected by hierarchy (n=44 pairs). B) Alluvial diagram depicting distribution of hierarchy classes from diagnosis to relapse. The width of each band reflects the number of patients transitioning from one class to another from diagnosis to relapse. C) Changes in malignant cell type abundance from diagnosis to relapse. Significance was evaluated using a Wilcoxon signed-rank test. D) Single-cell RNA-seq of diagnostic AML from van Galen et al (Cell 2019) compared to relapsed AML samples from Abbas et al (manuscript under review), classified, down sampled to 10,000 cells, and projected onto a common embedding using scArches with scANVI. E) Changes in overall LSPC abundance, PC1, and PC2 from diagnosis to relapse, stratified by cellular hierarchy at diagnosis. F) Patients with NPM1+FLT3-ITD (recurrently acquired at relapse) G) Patients with NPM1c+RAS, and NPM1c+FLT3-TKD (recurrently lost at relapse). H-I) Changes in clonal and cell-type composition from diagnosis to relapse. These are depicted for a patient with concordant shifts in both clonal composition and cell-type composition (H) as well as for a patient with minimal change in clonal composition and drastic changes in cell-type composition (I).

FIG. 4. Hierarchy composition in AML as a determinant of targeted therapy response. A) Correlation between cell type abundance and ex vivo drug sensitivity (−AUC) across 202 patients in BEAT-AML, wherein color and size represent the direction and magnitude of the correlation. Only correlations with p<0.05 are depicted, those with q<0.05 are marked with an asterisk. B) LinClass-7 (trained on PC2) captures the stem vs mature axis. C) Correlation with LinClass-7 identifies drugs targeting either primitive blasts or mature blasts from BEAT-AML (Nature 2018) and Lee et al (Nat Commun 2019). D) Venetoclax and Azacytidine target primitive AMLs (LinClass-7 high), MEK and MTOR inhibition targets mature AMLs (LinClass-7 low). E) Mean expression of CD33, the target of Gemtuzumab-Ozagomycin, in AML blast populations from scRNA-seq. Sensitivity values are depicted as scaled Area Above the Dose-Response Curve (AAC). F) Event-free survival and relapse-free survival of control patients (Daunorubicin+Cytarabine) vs GO patients (Daunorubicin+Cytarabine with Gemtuzumab-Ozagomycin) from the ALFA-0701 trial. Patients are stratified by LinClass-7 score, wherein high LinClass-7 denotes primitive disease while low LinClass-7 denotes mature disease.

FIG. 5. Cellular basis of drug response in preclinical studies. A) Experimental design of re-analyzed preclinical studies from the literature. Only studies of human AML with RNA-seq available before and after drug treatment were included in order to quantify changes in cell type composition. B) Schematic of re-analysis approach. Changes in the abundance of each cell type were quantified in each treatment condition, and treatments with significant changes in at least one cell type were used as input for dimensionality reduction with UMAP and subsequent clustering. C) Clustering of drug treatments on the basis of changes in cell type composition. D) Heatmap depicting cell type composition changes of drug treatments within each cluster. Purple denotes decreased abundance following treatment and green denotes increased abundance following treatment. E) Drug treatments that caused differentiation, on the basis of a significant decrease in PC2. F) Examples of the drug treatments targeting specific processes and the changes induced in the abundance of each cell type following treatment. G) Cellular composition changes following in vitro Selinexor treatment in NPM1 mutant AMLs from Brunelli et al, 2018. H) Mean expression of XPO1 (the target of Selinexor) and associated genes and pathways in AML blast populations from scRNA-seq. Geneset for the nuclear export pathway was obtained from GO Biological Pathways. I) Correlation between cell type abundance and ex vivo drug sensitivity in BEAT-AML. Correlations with p<0.05 are marked with an asterisk. J) LSPC-cycle abundance in primary AML samples treated with DMSO control or Selinexor in vitro (Treatment and RNA-seq from Brunelli et al, 2018). K) LSPC-cycle abundance in primary AML samples treated with DMSO control or Selinexor in vivo (Treatment from Etchin et al, 2015; RNA-seq from this study).

FIG. 6. Hierarchy-based stratification predicts in vivo drug responses of Fedratinib and CC90009. A) Mean expression of JAK2, the target of Fedratinib, in AML blast populations from scRNA-seq. B) Differences in cell-type abundance between full responders and partial/non-responders to Fedratinib. Red depicts enrichment in full responders and blue depicts enrichment in partial/non-responders. Significant differences (p<0.05) are marked with an asterisk. C) Xenograft responses to Fedratinib, stratified by leukemic hierarchy cluster. Bar plot depicts mean difference in engraftment in Fedratinib treated mice compared to Vehicle treated mice. Cell-type composition of each patient prior to treatment is depicted below each bar. D) GSEA of bulk RNA-seq from Primitive Fedratinib full-responders compared to partial/non-responders, assessing enriched for NPM1c and electron transport chain signatures. E) Mean expression of GSPT1, the target of CC90009, in AML blast populations from scRNA-seq. F) Differences in cell-type abundance between full responders and partial/non-responders to CC90009, represented as the −log(pvalue). Red depicts enrichment in full responders and blue depicts enrichment in partial/non-responders. Significant differences (p<0.05) are marked with an asterisk. G) Xenograft responses to CC90009, stratified by leukemic hierarchy cluster. Bar plot depicts mean difference in engraftment in CC90009 treated mice compared to Vehicle treated mice. Cell-type composition of each patient prior to treatment is depicted below each bar. H) GSEA of bulk RNA-seq from Primitive CC90009 full-responders compared to partial/non-responders, assessing enriched for NPM1c and electron transport chain signatures. I) Response to Fedratinib+CC90009 combination treatment. Patients are stratified by hierarchy and mean engraftment levels are depicted for each treatment condition. J) in vivo efficacy of Fedratinib, CC-90009, and Combination treatment of xenografted AML patient samples, stratified by patient hierarchy.

FIG. 7. Features of leukemia stem and progenitor cell populations from scRNA-seq. A-E) Diffusion map of re-annotated LSPC populations using feature weights from Self-Assembling Manifolds (SAM), depicting A) Patient source, B) prior cell type annotation, C) enrichment of LSC-specific genes from Ng et al 2016 and Shannon Diversity Index, D) scaled CDK6 expression and enrichment of the E2F3 regulon, and E) enrichment of E2F1 and CTCF regulons. F) Cell cycle status of Quiescent, Primed, and Cycling LSPCs. G) Enrichment of inflammatory signaling pathways and regulons in LSPCs. H) Transcription factor regulon activity inferred through pySCENIC specific to each LSPC. I) Silhouette score of original and new LSPC annotations calculated from three different embeddings, including PCA with SAM feature weights, UMAP with SAM feature weights, and UMAP with highly variable genes. J) Receiveroperator curves from classification of original and new LSPC annotations using SingleCellNet, a Random Forest-based classifier for single cell RNA-seq data trained from the top pairs of genes unique to each cell type.

FIG. 8. Benchmarking gene expression deconvolution approaches for AML. A) Correlation between observed and predicted cell-type frequencies of pseudo-bulk scRNA-seq mixtures from five deconvolution approaches. B) Reference correlation between malignant cell-types across scRNA-seq samples from 12 diagnostic AML patients. C) Observed correlation between malignant cell types from deconvolution analysis of 173 patients within the TCGA cohort, depicted for each deconvolution tool. MuSIC Direct was excluded due to multiple celltypes having a detection rate of zero in bulk RNA-seq. D) PCA plot of deconvoluted TCGA AML patients by cell-type composition, depicted for each deconvolution tool. Relative abundance of Quiescent, Primed, and Cycling LSPC are depicted, as these populations are highly correlated within the scRNA-seq data. E) Signature matrix for CIBERSORTx deconvolution. F-G) Correlation between observed transcriptomic profiles and synthetic transcriptomic profiles reconstructed based on predicted cell-type abundance from CIBERSORTx. Higher correlation suggests greater deconvolution confidence. These correlations are depicted for F) Deconvolution of AML cohorts using reference signatures from malignant AML populations compared to deconvolution with reference signatures from matched healthy populations. Immune cell types were also included for both approaches. G) RNA-seq compared to microarray of sorted LSC fractions. RNA-seq fractions were collected from a subset of the same biological samples as array fractions.

FIG. 9. Functional correlates of LSPC populations inferred through deconvolution. A) Logistic regression classifier for predicting engraftment potential in AML fractions trained on cell-type abundance and CD34/CD38 immunophenotype. The average importance of each feature (Shapley values) is depicted. B) Random Forest classifier for predicting engraftment potential in AML fractions trained on cell-type abundance and CD34/CD38 immunophenotype. The average importance of each feature (Shapley values) is depicted. C-D) Relative abundance of Quiescent and Primed LSPC within LSC+ (engrafting) and LSC− (nonengrafting) fractions. As nearly all available CD34+CD38− fractions were LSC+, these are depicted with C) CD34+/CD38− fractions excluded as well as with D) all CD34+ fractions excluded. E) Relative abundance of Quiescent and Primed LSPC in Leucegene patients with low, medium, and high bulk LSC frequencies, as defined from Pabst et al (Blood 2016).

FIG. 10. Biological and clinical correlates of AML hierarchies. A) Correlation between cell-type abundance and FPKM-normalized miRNA expression in TCGA, wherein color and size represent the direction and magnitude of the correlation. Only correlations with p<0.05 are depicted, and correlations with FDR<0.05 are noted with an asterisk. For visualization top 5 correlated miRNAs were selected for each malignant cell-type. B) Correlation between cell-type abundance and Beta values of methylation probes from TCGA, depicting global methylation patterns associated with different malignant cell-types. Methylation probes were downsampled to 50,000 for visualization purposes. C-D) Correlation between cell-type abundance and clinical features in TCGA. Only correlations with p<0.05 are depicted, and correlations with FDR<0.05 are noted with an asterisk. These are depicted for C) malignant cell-types as well as D) immune cell-types. E) Ridge plots depicting FAB categorizations from TCGA, BEATAML, and Leucegene across PC1 (Stem to GMP) and PC2 (Stem to Mature). F) FAB categories from TCGA, BEAT-AML, and Leucegene projected by cellular hierarchy. G) Correlation between LSC17 score and cell type abundance across TCGA, BEAT-AML, and Leucegene cohorts. H-I) Overall survival of patients grouped by hierarchy cluster, in G) chemotherapy treated TCGA patients and H) BEAT-AML patients, with and without exclusion of patients receiving transplants.

FIG. 11. Genomic correlates of AML hierarchies. A) Density plots depicting all mutation combinations along the Stem vs Mature axis (PC2). B) Density plots depicting all mutation combinations along the Stem vs GMP axis (PC1). C) Density plots depicting all cytogenetic alterations along the Stem vs Mature axis (PC2). D-E) Impact of DNMT3A R882 mutations compared to other DNMT3A mutations on leukemic hierarchy organization along the Stem vs Mature axis (PC2). D) Boxplot comparing PC2 of DNMT3A R883 mutant AML compared to other DNMT3A mutations within the context of each mutational combination. E) Density plot depicting PC2 of mutational combinations with DNMT3A R882 and with other DNMT3A mutations.

FIG. 12. AML hierarchies underlie survival and induction failure in pediatric AML. A) Pediatric AML samples from the TARGET-AML cohort (n=287 patients) are projected by cellular hierarchy and classified based on hierarchy clusters defined from adult AML cohorts. B) Overall survival across hierarchy clusters in pediatric AML. C) Event-free survival across hierarchy clusters in pediatric AML. D) Differences in cell-type abundance between pediatric AML patients achieving complete remission (CR) compared to those failing induction therapy. E) Association between cell-type abundance and induction failure in four independent studies spanning Pediatric (TARGET-AML) and Adult (Chiu et al, Herold et al, BEAT-AML) AML. Green indicates higher abundance in induction failure patients while purple indicates lower abundance in induction failure patients. Differences with a significance of p<0.10 are noted with an asterisk.

FIG. 13. Changes in cellular composition from diagnosis to relapse. A) Celltype composition of 44 matched diagnosis and relapse pairs. Top row depicts cellular composition at diagnosis while the bottom row depicts cellular composition at relapse. Samples from the same patient are aligned vertically. B) Changes in each malignant blast population from diagnosis to relapse. Changes in PC1 and PC2 are also depicted. E-H) Evolution of paired diagnosis and relapse AML samples depicted through shifts in cellular hierarchies, evolution of genetic subclones, and changes in cell-type composition. E) Patient 303642, in which significant genetic evolution is accompanied by a dramatic shift in cellular hierarchy from GMP to primitive. F) Patient 4, in which a loss of monocytic blasts is accompanied by a modest decrease in the size of an NRAS bearing clone. G) Patient 1019, in which replacement of an NRAS and IDH2 positive clone with an IDH1 positive clone is associated with a modest shift in cellular hierarchy. H) Patient 150288, in which extensive linear genetic evolution is not associated with any appreciable change in cell type composition.

FIG. 14. Biological correlates of the LinClass-7 score. A) Features and corresponding weights comprising the LinClass-7 score. B) Pearson correlation between LinClass-7 and PC2 in TCGA, Leucegene, and BEAT-AML. C) Correlation of cell type abundance with LinClass-7, LSC17, and each principal component across TCGA, Leucegene, and BEAT-AML cohorts. D) LinClass-7 scores split by FAB morphology across TCGA, BEAT-AML, and Leucegene. E) LinClass-7 scores by bulk LSC frequency in Leucegene. F) LinClass-7 scores by functionally defined relapse origin and inferred relapse origin. G) Association between LinClass-7 and LSC-17 across TCGA, BEAT-AML, and Leucegene cohorts. Patients are colored by their hierarchy cluster.

FIG. 15. Literature screen to identify cellular targets of differentiation-inducing drugs. A) Changes in cell type composition following drug treatment from preclinical studies in human AML. Green depicts an increase in cell type abundance and purple depicts an decrease in cell type abundance. B) UMAP coordinates for each drug treatment condition depicting changes to each cell type, as well as tissue source (Primary vs Cell Line), MLL translocation status, and drug target.

FIG. 16. Applying the leukemic hierarchies framework for pre-clinical studies in AML. A) Leukemic hierarchies of primary patient samples prior to in vivo treatment with Fedratinib and/or CC90009, categorized by cluster and projected onto the reference distribution. B) Relative abundance of each malignant cell type in each patient. C) Xenograft responses to Fedratinib among Primitive AMLs. Bar plot depicts mean difference in engraftment in Fedratinib treated mice compared to Vehicle treated mice. NPM1c and FLT3-ITD genotype. D-E) GSEA of bulk RNA-seq from Primitive Fedratinib full-responders compared to partial/nonresponders, showing that Fedratinib Responders are enriched for (D) NPM1c signatures and (E) electron transport chain machinery. F) Xenograft responses to CC90009 among Primitive AMLs. Bar plot depicts mean difference in engraftment in CC90009 treated mice compared to Vehicle treated mice. NPM1c and FLT3-ITD genotype. G-H) GSEA of bulk RNA-seq from Primitive CC90009 full-responders compared to partial/non-responders, showing that CC90009 Non-Responders are enriched for (G) NPM1c signatures and (H) electron transport chain machinery. I-J) GSEA of LSPCs from NPM1c patients compared to LSPCs non-NPM1c patients from scRNA-seq, stratified by each subpopulation. In each LSPC subpopulation, NPM1c is associated with enrichment in Electron Transport Chain machinery (I) and specifically genes involved in Complex I assembly (J).

FIG. 17. LinClass-7 is predictive rather than prognostic. A) Overall survival of patients in the TCGA and BEAT-AML cohorts grouped into “High” or “Low” based on median splits of patient scores for LSC17 and LinClass-7. B) Correlations of LSC17 and LinClass-7 scores with drug sensitivities from two drug screens (Tyner et al 2018, Lee et al 2018) performed on primary AML samples.

FIG. 18. Drug sensitivity predictions by LinClass-7 are not matched by LSC17. Ex vivo drug sensitivity to Venetoclax, Navitoclax, Mubritinib, and Azacytidine of primary patient samples grouped into “High” or “Low” based on median splits of patient scores for (A) LinClass-7 and (B) LSC17.

FIG. 19. LinClass-7 as a companion score for LSC17. A) LSC17 and LinClass-7 scores of 864 AML patients by RNA-seq. Patients belonging to each hierarchy cluster (Primitive, Intermediate, GMP, Mature) are also depicted. (B) LSC17 and LinClass-7 scores measured through a 17-gene NanoString test. Normalized NanoString-derived LSC17 and LinClass-7 scores from 306 patients at Princess Margaret Hospital are depicted.

### DESCRIPTION

In the following description, numerous specific details are set forth to provide a thorough understanding of the invention. However, it is understood that the invention may be practiced without these specific details.

The treatment landscape of AML is evolving with many promising targeted therapies in clinical translation, yet patient responses remain heterogeneous and reliable biomarkers for tailoring treatment are lacking. To develop a new approach for characterizing disease heterogeneity and therapy response, we deconvoluted bulk RNA profiles from large AML patient cohorts using scRNA-seq reference profiles of AML cells spanning each level of the leukemia cell hierarchy. The composition of leukemia cell hierarchies from over 1000 individual patients was interrogated and found to converge into four overall classes, each correlated to discrete functional and genomic properties. Primitive hierarchy composition correlated with induction failure and poor survival outcomes, and hierarchies from samples collected at diagnosis frequently transitioned to Primitive at relapse. Critically, hierarchy composition predicted response to a wide range of investigational drugs, demonstrating the potential to facilitate therapy selection. Together, our approach constitutes a novel framework for advancing precision medicine in AML.

In an aspect, there is provided a method of predicting treatment response to a drug in a patient with leukemia, wherein the drug had been predetermined to preferentially target either primitive or mature leukemic cells, the method comprising: determining the expression level of at least 3 genes in a test sample from the subject selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, and GPR56; calculating a primitiveness score comprising the weighted sum expression of each of the at least 3 genes; and either predicting that the patient will be sensitive to treatment by the drug if (i) the drug preferentially targets primitive leukemic cells and the calculated primitiveness score is high in reference to a control cohort of leukemia patients; or (ii) the drug preferentially targets mature leukemic cells and the calculated primitiveness score is low in reference to a control cohort of leukemia patients; or predicting that the patient will be resistant to treatment by the drug if (i) the drug preferentially targets primitive leukemic cells and the calculated primitiveness score is low in reference to a control cohort of leukemia patients; or (ii) the drug preferentially targets mature leukemic cells and the calculated primitiveness score is high in reference to a control cohort of leukemia patients.

The term “subject” as used herein refers to any member of the animal kingdom, preferably a human being and most preferably a human being that has AML or that is suspected of having AML.

The term “test sample” as used herein refers to any fluid, cell or tissue sample from a subject which can be assayed for biomarker expression products and/or a reference expression profile, e.g. genes differentially expressed in subjects with AML according to survival outcome. In an embodiment, the sample comprises WBCs obtained from peripheral blood (PB) or bone marrow (BM).

The phrase “determining the expression of genes” as used herein refers to determining or quantifying RNA or proteins or protein activities or protein-related metabolites expressed by the biomarkers. The term “RNA” includes mRNA transcripts, and/or specific spliced or other alternative variants of mRNA, including anti-sense products. The term “RNA product of the biomarker” as used herein refers to RNA transcripts transcribed from the biomarkers and/or specific spliced or alternative variants. In the case of “protein”, it refers to proteins translated from the RNA transcripts transcribed from the biomarkers. The term “protein product of the biomarker” refers to proteins translated from RNA products of the biomarkers.

The term “level of expression” or “expression level” as used herein refers to a measurable level of expression of the products of biomarkers, such as, without limitation, the level of micro-RNA, messenger RNA transcript expressed or of a specific exon or other portion of a transcript, the level of proteins or portions thereof expressed of the biomarkers, the number or presence of DNA polymorphisms of the biomarkers, the enzymatic or other activities of the biomarkers, and the level of specific metabolites.

As used herein, the term “control” refers to a specific value or dataset that can be used to prognose or classify the value e.g expression level or reference expression profile obtained from the test sample associated with an outcome class.

A person skilled in the art will appreciate that a number of methods can be used to detect or quantify the level of RNA products of the biomarkers within a sample, including arrays, such as microarrays, RT-PCR (including quantitative RT-PCR), nuclease protection assays and Northern blot analyses. For example, biomarkers may be measured using one or more methods and/or tools, including for example, but not limited to, Taqman (Life Technologies, Carlsbad, Calif.), Light-Cycler (Roche Applied Science, Penzberg, Germany), ABI fluidic card (Life Technologies), NanoString® (NanoString Technologies, Seattle, Wash. and as described in U.S. Pat. No. 7,473,767), NANODROP® technology (Thermo Fisher Scientific (Wilmington, Del.), fluidic card, and the like. The person of skill in the art will recognize such other formats and tools, which can be commercially available or which can be developed specifically for such analysis. Regarding nanostring specifically, it is also known to use synthetic oligonucleotides as a control in each nanostring cartridge to minimize inter-cartridge batch effects between runs.

In some embodiments, the calculated primitiveness score is high if it is higher than the median score of the control cohort of leukemia patients and the calculated primitiveness score is low if it is lower than the median score of the control cohort of leukemia patients.

In some embodiments, the leukemia is acute myeloid leukemia.

In a preferred embodiment, the at least 3 genes consists of DNMT3B, LAPTM4B, CDK6, CPXM1, NGFRAP1, CD34, and GPR56.

In some embodiments, the method further comprises treating the patient with the drug if the patient has been predicted to be sensitive to treatment by the drug according to the patient's primitiveness score.

In some embodiments, the drug preferentially targeting primitive leukemia cells is Selinexor, Venetoclax, Erlotinib, GSK-1838705A, Gefitinib, Canertinib (CI-1033), Pelitinib (EKB-569), PHA-665752, Barasertib (AZD1152-HQPA), Palbociclib, Sorafenib, NVP-ADW742, NF-kB Activation Inhibitor, Bay 11-7085, Lenalidomide, Afatinib (BIBW-2992), SR9011, KU-55933, KW-2449, Roscovitine (CYC-202), LY-333531, NVP-TAE684, Vandetanib (ZD6474), Pazopanib (GW786034), Vargetef, Dovitinib (CHIR-258), Vatalanib (PTK787), Vemurafenib (PLX-4032), Tipifarnib, PLX-4032, BAY 11-7082, Lomustine, MLN8237, PLX-4720, Azacitidine, 5-lodotubercidin, NVP-TAE-684, Mubritinib, Decitabine, BI-2536, NVP-LDE-225, Tosedostat, SB 218078, Flavopiridol, Melphalan, AS101, BSI-201, ABT-263, Fenretinide, ARQ-197, Tozasertib, BAY 11-7085, PD0332991, Topotecan HCl, Pravastatin, Etoposide, Vinblastine sulfate, GDC-0449, Pemetrexed, TG-101348, PIK-75, Acrichine, AS-605240, Gemcitabine HCl, ABT-888, XL-147, Bexarotene, Crizotinib, Erlotinib HCl, Etodolac, Otava 7015980251, BIBW 2992, FTI-276, Irinotecan HCl, BML 277, Vincristine sulfate, Arsenic trioxide, PF-04449913, GF109203X, or CC-90009.

In some embodiments, the drug preferentially targeting mature leukemic cells is GW-2580, JNJ-28312141, INK-128, Staurosporine, Idelalisib, MK-2206, PRT062607, Cediranib, Linifanib, Go6976, DasaUnib, PLX-4720, CI-1040, 17-AAG, Tandutinib, Rapamycin, PKI-587, Everolimus, Temsirolimus, Panobinostat, Selumetinib (AZD6244), GDC-0941, Nilotinib, BEZ235, MK-2206, SNS-032 (BMS-387032), Flavopiridol, TG100-115, Trametinib (GSK1120212), Cediranib (AZD2171), Bortezomib (Velcade), or Fedratinib.

In an aspect, there is provided a composition comprising a plurality of isolated nucleic acid sequences, wherein each isolated nucleic acid sequence hybridizes to: (a) the mRNA of at least 3 genes selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, GPR56; and/or (b) a nucleic acid complementary to a), wherein the composition is used to measure the level of expression of the at least 3 genes. In some embodiments, the at least 3 genes consists of DNMT3B, LAPTM4B, CDK6, CPXM1, NGFRAP1, CD34, and GPR56.

The term “nucleic acid” includes DNA and RNA and can be either double stranded or single stranded.

The term “hybridize” or “hybridizable” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C., followed by a wash of 2.0×SSC at 50° C. may be employed.

The term “probe” as used herein refers to a nucleic acid sequence that will hybridize to a nucleic acid target sequence. In one example, the probe hybridizes to the RNA biomarker or a nucleic acid sequence complementary thereof. The length of probe depends on the hybridization conditions and the sequences of the probe and nucleic acid target sequence. In one embodiment, the probe is at least 8, 10, 15, 20, 25, 50, 75, 100, 150, 200, 250, 400, 500 or more nucleotides in length.

The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent.

The exact length of the primer will depend upon factors, including temperature, sequences of the primer and the methods used. A primer typically contains 15-25 or more nucleotides, although it can contain less or more. The factors involved in determining the appropriate length of primer are readily known to one of ordinary skill in the art.

In an aspect, there is provided an array comprising, for each of at least 3 genes selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, GPR56, one or more polynucleotide probes complementary and hybridizable thereto. In some embodiments, the at least 3 genes consists of DNMT3B, LAPTM4B, CDK6, CPXM1, NGFRAP1, CD34, and GPR56

In an aspect, there is provided a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the method described herein.

In an aspect, there is provided a computer implemented product for predicting treatment response to a drug in a patient with leukemia, wherein the drug had been predetermined to preferentially target either primitive or mature leukemic cells, the computer implemented product comprising: (a) a means for receiving values corresponding to a subject expression profile comprising at least 3 genes from the subject selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, and GPR56; (b) a database comprising a reference expression profile representing a control, wherein the subject expression profile and the reference profile each have at least one value representing the expression level of at least 3 genes selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, GPR56; (c) a means for calculating a primitiveness score comprising the weighted sum expression of each of the at least 3 genes; and either (d) a means for outputting a prediction that that the patient will be sensitive to treatment by the drug if (i) the drug preferentially targets primitive leukemic cells and the calculated primitiveness score is high in reference to a control cohort of leukemia patients; or (ii) the drug preferentially targets mature leukemic cells and the calculated primitiveness score is low in reference to a control cohort of leukemia patients; or (e) a means for outputting a prediction that the patient will be resistant to treatment by the drug if (i) the drug preferentially targets primitive leukemic cells and the calculated primitiveness score is low in reference to a control cohort of leukemia patients; or (ii) the drug preferentially targets mature leukemic cells and the calculated primitiveness score is high in reference to a control cohort of leukemia patients.

In an aspect, there is provided a method for selecting a drug in a patient with leukemia, wherein the drug had been predetermined to preferentially target either primitive or mature leukemic cells, the method comprising the method of any one of claims 1-7, and further comprising selecting the drug if the patient had been predicted to be sensitive to treatment by the drug according to the patient's primitiveness score.

In an aspect, there is provided a drug for use in the treatment of leukemia in a patient, wherein the patient had been determined to be sensitive to the drug by the method described herein.

In an aspect, there is provided a use of a drug in the preparation of a medicament for the treatment of leukemia in a patient, wherein the patient is determined to be sensitive to the drug by the method described herein.

The advantages of the present invention are further illustrated by the following examples. The examples and their particular details set forth herein are presented for illustration only and should not be construed as a limitation on the claims of the present invention.

### Examples

In this study, we derived reference profiles of distinct AML cell types from scRNA-seq data and applied gene expression deconvolution on bulk AML transcriptomes to characterize the cellular hierarchies of more than one thousand AML patients. The composition of these leukemic hierarchies converge into four main classes that are correlated to discrete functional and genomic properties. Hierarchy composition changed throughout disease progression. Critically, variation in hierarchy composition was associated with differences in response to chemotherapy as well as a broad range of targeted therapies. This approach to characterizing AML heterogeneity integrates genomic and functional models of AML into a novel framework for understanding disease biology and predicting drug response.

### Methods and Materials

### Patient Samples

All biological samples were collected with informed consent according to procedures approved by the Research Ethics Board of the University Health Network (UHN; REB #01-0573-C) and viably frozen in the PM Leukaemia Bank. No statistical methods were used to predetermine sample size. The investigators were not blinded to allocation during experiments and outcome assessment.

### RNA Sequencing and Pre-Processing

RNA was extracted from bulk peripheral blood mononuclear cells using an RNeasy Micro Kit (Qiagen). Libraries were constructed using SMART-Seq (Clonetec). A paired-end 50-base-pair flow-cell lane Illumina HiSeq 2000 yielded an average of 240 million aligned reads per sample. To align RNA-seq reads from samples used in selinexor and fedratinib treatments, Illumina paired-end sequence data were analyzed with BWA/v0.6 alignment software with option (-s) to disable Smith-Waterman alignment. Reads were mapped onto GRCh37-lite reference genome and exon-exon junction reference whose coordinates were defined based on transcript annotations in Ensembl/v59. Reads with mapping quality <10 were discarded and duplicate reads were tagged using the Picard's MarkDuplicates program. JAGuaR 2.1 was used to incorporate reads spanning multiple exons into the alignment by introducing large alignment gaps. All transcripts of a given gene were collapsed into a single gene model such that exonic bases were the union of exonic bases that belonged to all known transcripts of the gene. Read counts and subsequently RPKM counts were obtained by counting the fraction of each read that overlapped with an exonic region for that gene. To align RNA-seq reads from functionally annotated LSC fractions, sequence data was aligned against GRCh38 and transcript sequences downloaded from Ensembl build 90 using STAR 2.5.2a. Default parameters were used except for the following: “-chimSegmentMin 12 -chimJunctionOverhangMin 12 -alignSJDBoverhangMin 10 -alignMatesGapMax 100000 -alignIntronMax 100000 -chimSegmentReadGapMax parameter 3 -alignSJstitchMismatchNmax 5 −1 5 5.” Counts were obtained using HTSeq v0.9.1.

### Re-Clustering of Leukemia Stem and Progenitor Cells (LSPCs)

Single cell RNA-sequencing data from 12 AML patients at diagnosis was obtained from van Galen et al³² (GSE116256). scRNA-seq count data was normalized using the R package ‘scran’⁷², log-transformed with an offset value of 1, and scaled. Malignant AML cells labeled as “HSC-like” and “Prog-like” (hereafter LSPCs) from the original study were subject to re-analysis using the Self-Assembling Manifolds (SAM) algorithm³⁴. SAM was applied individually to the four patient samples with the highest number of LSPCs to assign weights to each gene based on how well they can demarcate emerging transcriptomic states. Feature weights for each gene were averaged across the four samples and subsequently applied to LSPCs from all 12 patients. No batch correction was applied. Using the “scanpy” package⁷³, weighted expression data was subject to dimensionality reduction and neighbourhood detection based on the cell-cell correlation. The diffusion map embedding⁷⁴ was used for visualization. Leiden clustering⁷⁵ was performed with a resolution of 0.15 to identify three clusters of LSPCs shared across the patient samples (Re-annotated LSPC labels—data not shown).

### Evaluation of LSPC Clustering

To evaluate the new cluster assignments, silhouette scores were calculated for the new and prior LSPC classifications for each of three separate embeddings: PCA from SAM weights, UMAP from SAM weights, and UMAP from highly variable genes. For each embedding, the average silhouette scores for each patient sample from the new classification were compared to those from the prior classification through paired t-tests. As an alternative approach to evaluate the new cluster assignments, cell-type classifiers were built and evaluated for the new and prior classifications using the R package “SingleCellNet”⁷⁶. For each classification, scran normalized gene expression values were used as input and 800 cells from each malignant cell type were used as a training set. For each cell type, paired products of the top 25 genes for each cell type were calculated and the 50 top gene pairs for each cell type were used to train the Random Forest based model with nTrees=1000. Models trained on the new and prior cell-type classification were subsequently evaluated on a held-out dataset of at least 250 remaining cells for each cell-type.

### Regulon Analysis and Signature Enrichment

To infer transcription factor (TF) regulon activity in scRNA-seq data, regulon analysis was performed using SCENIC⁷⁷. The Docker image of pySCENIC was run as per the guidelines from Van de Sande et al⁷⁸: log-transformed counts from malignant AML cells were used as the input and candidate transcription factors were identified using a list of human transcription factors from Lambert et al⁷⁹, with default parameters. To prune putative TF-target links within each regulon using annotations of TF motifs, CisTarget was applied using databases of known human TF motifs annotated at 500 bp, 5 kb, and 10 kb of transcriptional start sites. Drop-out masking was also applied during this step. Enrichment of refined TF regulons was inferred using AUCell, and enrichment scores were scaled for visualization.

Characterization of scRNA-seq AML Populations

For biological characterization of the re-annotated malignant cell types, single cell enrichment scores of hallmark genesets as well as custom genesets from Ng et al (LSC+ AML fractions)²⁶ and Xie et al (S1PR3 overexpression in LT-HSCs)³⁶ were calculated using AUCell⁷⁷. Cell cycle status was determined using the original annotations from van Galen et al³², in which cell cycle scoring and classification was performed. Shannon diversity of single cell transcriptomes was calculated from raw count data using the python package “skbio” after down-sampling each cell to 1,000 UMIs.

### Gene Expression Deconvolution

Raw gene expression counts from 13653 cells belonging to any of seven malignant populations (LSPC-Quiescent, LSPC-Primed, LSPC-Cycle, GMP-like, ProMono-like, Mono-like, cDC-like) or seven non-leukemic immune populations (T, CTL, NK, B, Plasma, and wild-type Monocyte and cDCs) were used as input for signature matrix generation with CIBERSORTx⁴⁰. Default settings were used with the exception of the minimum expression parameter which was set to 0.25. Deconvolution was performed on TPM-normalized bulk RNA-seq data using S-mode batch correction and Absolute mode. Due to differences in S-mode batch correction performance between the CIBERSORTx web portal and the CIBERSORTx Docker image, we exclusively used the web portal for our analyses. For downstream analysis, the abundance of the seven malignant populations were normalized to a sum of 1, wherein the score for each population represents the estimated proportion of all leukemic cells. For bulk RNA-seq samples composed entirely of leukemic blasts (cell lines or sorted primary samples), a second signature matrix with seven malignant populations and no immune populations was used.

### Comparison of Deconvolution Approaches

For deconvolution with DWLS⁴¹, a single-cell signature matrix was generated using MAST⁸⁰ for each cell type using default settings from the DWLS script. DWLS was then applied to pseudo-bulk and TPM-normalized TCGA RNA-seq data using default settings. Deconvolution with Bisque⁴² was applied to AML pseudo-bulk and unnormalized TCGA RNA-seq data, following package guidelines and using default settings. Wild-type Monocytes and cDCs were removed to improve performance. Deconvolution with MUSIC⁴³ was applied to the pseudobulk data and unnormalized TCGA RNA-seq data, as per tool guidelines. This was performed in two different ways: direct and recursive. Direct deconvolution involves calculating cell type abundance of each population directly. In order to deal with issues arising from co-linearity, recursive deconvolution was also applied which first calculated the abundance of four groups of cell types: LSPC (LSPC-Quiescent, LSPC-Primed, LSPC-Cycle), GMP (GMP-like), Mature (ProMono, Mono, cDC-like), and Immune (T, B, NK, CTL, Plasma, cDC, Monocyte), and subsequently calculated the abundance of each individual cell type from each group.

To evaluate deconvolution performance, pseudo-bulks profiles were generated using count data from single cells on a per-patient basis and normalized into counts per million (CPM). These were used for deconvolution from each approach and the correlation between observed and predicted abundance was calculated for each cell type. To assess how each deconvolution approach preserves relationships between each malignant cell type and deals with co-linearity, we generated a dendrogram on the basis of correlation between each cell type across the 12 patients. We repeated this analysis for deconvolution results from TCGA to see how well these relationships were preserved when applied to bulk RNA-seq data.

To assess confidence of deconvolution results from CIBERSORTx, we used the correlation metric provided in the CIBERSORTx output, which represents the agreement between the original bulk transcriptome and the ‘synthetic’ transcriptome constructed from combining the reference signatures of each cell type at their estimated frequencies. To compare deconvolution confidence between malignant and healthy reference signatures, reference signatures from healthy hematopoiesis were derived from healthy bone marrow data from van Galen et al, using profiles of the following cell types: HSC, Prog, GMP, ProMono, Mono, cDC, T, B, NK, CTL, Plasma.

### Clinical AML Datasets

Publicly available clinical RNA-seq datasets (data not shown) were used for deconvolution analysis. All gene expression data was subject to TPM normalization prior to deconvolution with CIBERSORTx. Clinical and mutational data was extracted from the GDC Data Portal for TCGA (https://portal.gdc.cancer.gov/projects/TCGA-LAML) and from supplemental materials in Tyner et al 49 for BEAT-AML. For the Leucegene cohort, clinical and mutational annotations were extracted from supplemental materials of 13 papers 45.81-92 and linked based on sample ID.

### Mapping and Clustering AML Hierarchy Composition

To map AML patients based on the composition of their leukemic hierarchies, only deconvolution results pertaining to malignant AML populations were used. In these cases, estimated abundances from malignant populations were normalized to 1, such that the value associated with each cell type represents the proportion of total malignant blasts that it constitutes. Patients from TCGA, BEAT-AML, and Leucegene were used. Known post-treatment samples from BEAT-AML were excluded from clustering analysis. PCA was performed on the normalized malignant cell type compositions of these patients. Neighbours were calculated using euclidean distance with a local neighborhood size of 30. To determine the optimal number of clusters, the packages “NbClust” was used to calculate 30 clustering metrics for values of k from 2 to 10, and k=4 was selected by majority rule. Leiden clustering was subsequently performed at a resolution of 0.4 to obtain four hierarchy clusters (cluster assignments, hierarchy compositions, and genomic annotations for TCGA, BEAT-AML, and Leucegene—data not shown).

To project hierarchies onto the reference map from the three AML cohorts (TCGA, BEAT-AML, Leucegene), normalized malignant cell type abundances from the query dataset was combined with the reference dataset, and batch correction was applied using ComBat. Following this, the ingest function from scanpy was used to project the batch corrected query dataset onto the principal components of the batch corrected reference dataset and assign cluster labels.

### Survival Analysis

Overall survival (OS) was defined as the time from diagnosis until death or last follow-up. Differences in OS between hierarchy classes were evaluated using Mantel-Cox Log-Rank tests using the R package “survival”, and survival curves for each cluster were visualized using Kaplan-Meier plots using the R package “survminer”.

### MiRNA-Seq and Methylation Analysis

MiRNA-seq and methylation (HM450) profiles from TCGA patients were acquired from Firebrowse (http://firebrowse.org/?cohort=LAML) miRNA-seq expression values were TPM normalized, and beta values were used to depict methylation values at each probe. To depict associations between cell types and global methylation patterns, methylation data was randomly down-sampled to 50,000 probes.

### Mutation Analysis

Cytogenetic and driver mutation annotations from TCGA, BEAT-AML, and Leucegene were used to correlate hierarchy composition with genomic profiles. Mutation combinations between driver mutations were identified and all combinations present in at least 5 patients were retained and visualized along hierarchy axes PC1 and PC2 using the R package “ggridges”. Due to missing variant allele frequency (VAF) information in an appreciable subset of mutation calls from genomic annotations, samples were considered mutated as long as the mutation was called. This analysis was repeated exclusively using mutation calls where VAF>0.25 to confirm that the observed trends remained the same.

### Clonal Evolution Analysis

Clonal analysis of paired diagnosis and relapse samples from four independent cohorts was performed using annotated single nucleotide variant calls derived from targeted sequencing⁵⁶, whole exome sequencing⁵⁷, or whole genome sequencing^{21,58} data. Genetic clones were identified using PhyloWGS⁹³, selecting the phylogenetic tree with the highest log likelihood (LLH) value. In cases of tied LLH values, the simplest tree with the most representative branching patterns among the top candidates was manually selected. Graphical representations of evolution of genetic clones were depicted using the R package “Fishplot”⁹⁴ while representations of changes in cell type composition were depicted using the R package “ggAlluvial”.

scRNA-Seq Classification in Relapsed AMLs

scRNA-seq profiles of blast cells from 8 relapsed AML patient samples were obtained from Abbas et al (in press). To project these cells onto our cell types defined from diagnostic AML samples from van Galen et al³², we used a transfer learning approach implemented through the scANVI⁹⁵ and scArches⁹⁶ packages. First, semi-supervised dimensionality reduction was performed with scANVI using unnormalized scRNA-seq data from diagnostic AML samples filtered for 3000 variable genes with malignant cell type annotations and patient batch as a covariate. For scANVI, an initial unsupervised neural network was trained over 500 epochs with patience for early stopping set to 10 epochs, followed by a semi-supervised neural network incorporating cell type annotations that was trained over 200 epochs with a patience of 10 epochs. Transfer learning with scArches was subsequently applied to update the scANVI neural network using scRNA-seq data from the relapsed AML samples, and training was performed over 500 epochs with a patience of 10 epochs. The updated model was subsequently applied to both diagnostic and relapsed AML samples to generate a shared latent representation, and this latent representation was used for further dimensionality reduction with UMAP. For visualization purposes, the diagnostic and relapsed AML data were each subsampled to 10,000 cells.

Association with Drug Sensitivity

Ex vivo drug response in BEAT-AML samples was measured through the Area Under the dose-response Curve (AUC) metric, wherein a low AUC corresponds to sensitivity while a high AUC corresponds to resistance. AUC values were scaled and multiplied by −1 to represent sensitivity in each treatment condition. Pearson correlation was used to measure association between cell type abundance and drug sensitivities, following recommendations from a benchmarking study by Smirnov et al⁹⁷. Associations were depicted using the R package “corrplot”, and drug sensitivity volcano plots were generated using the R package “EnhancedVolcano”. For associations of cell type abundance with clinical and biological features, absolute cell type abundance was always used and pearson correlations were calculated unless otherwise specified.

### LinClass-7 Score Derivation

To derive the LinClass-7 score, logCPM-normalized expression of 16 genes from the LSC17 assay were used as input features for LASSO regression: DNMT3B, GPR56, NGFRAP1, CD34, DPYSL3, SOCS2, MMRN1, KIAA0125, EMP1, NYNRIN, LAPTM4B, CDK6, AKR1C3, ZBTB46, CPXM1, ARHGAP22. The 17th gene, C19orf77, was excluded due to lack of expression data in the Leucegene cohort. LASSO regression was performed on negative PC2 (high in Primitive and low in Mature) with leave-one-out cross validation using the LassoCV function from scikit-learn with a path length of 0.1. Patients from TCGA and Leucegene were combined into a training set, and patients from BEAT-AML were used as a validation set to evaluate the strength of the association between LinClass-7 and PC2.

### Literature Screen for Drug-Treated RNA-Seq Datasets

To identify RNA-seq datasets collected from AML samples before and after drug treatment, Applying the search terms “Acute Myeloid Leukemia” and “AML” with the “Homo sapien” and “RNA-sequencing” flags on Gene Expression Omnibus (GEO) and ArrayExpress, we identified 95 datasets posted before Jun. 17, 2021. From these, 53 were inhibitors that met the inclusion criteria of human AML samples with available RNA-sequencing data collected before and after drug treatment. Datasets with only differential expression results or Bigwig files were excluded. Datasets with less than three samples in each treatment group were also excluded, resulting in a total of 47 datasets included in the final analysis. Each dataset was processed and underwent TPM normalization and deconvolution with CIBERSORTx using a signature matrix of seven malignant cell types (LSPC-Quiescent, LSPC-Primed, LSPC-Cycle, GMP-like, ProMono-like, Mono-like, cDC-like). For quality control among cell line samples, the deconvolution correlation values from each sample across every dataset were compared and the jenks natural breaks algorithm was employed to identify cutoffs demarcating low, medium, and high correlation bins. Cell line samples classified as “low-correlation” with a correlation value below 0.437 were excluded from further analysis, leaving 42 datasets spanning 153 treatment conditions.

Quantifying Hierarchy Composition Changes after Drug Treatment

Relative changes in cell type abundance in each treatment condition were evaluated using Wilcoxon rank-sum tests for technical replicates or Wilcoxon signed-rank tests for biological replicates with paired treatment conditions. For dimensionality reduction with UMAP, we focused exclusively on changes in cell type abundance where the p-value was <0.05 to emphasize the key changes in cell type composition induced by each drug, resulting in 125 treatment conditions spanning 38 studies. Absolute log p-values were used to represent the magnitude of shift in cell type abundance, and cell type changes where p>0.05 were assigned a magnitude of zero. We then applied UMAP with the following parameters (n_neighbors=13, min_dist=0.05) to generate the final representation, and leiden clustering was applied with a resolution of 1. Cell type composition changes for treatment conditions were visualized with the R package “ComplexHeatmap” (data not shown).

### Fedratinib and CC90009—Hierarchy Classification

Using normalized malignant cell type composition data for 46 patient samples used for in vivo fedratinib or CC-90009 treatment, dimensionality reduction was performed and clustering was assigned using the leiden algorithm with a resolution of 0.7, yielding three clusters: Primitive, Mature, Intermediate/GMP. Owing to an under-representation of engrafting samples with GMP hierarchies, we did not attempt to divide the Intermediate/GMP cluster into Intermediate and GMP groups. Samples were subsequently projected on the reference map for visualization and confirmation of cluster assignments.

Fedratinib and CC90009—Response Classification

Patient samples were classified into response categories by comparing the relative reduction (RR) of AML engraftment in drug-treated mice versus vehicle-treated mice, as per Galkin et al 98. RR was calculated as: ((mean % engraftment in control mice)−(mean % engraftment in drug treated mice))/(mean % engraftment in control mice). Patient samples were classified as Responders if RR in the injected femur (Right Femur, RF) was >50%, classified as Partial Responders if we observed 20 to 50% RR in the RF or >20% in the non-injected femur (Bone Marrow, BM) only, and classified as Non-Responders if there was no statistically significant difference in engraftment levels between control- and drug-treated mice, or if RR was <20% in both RF and BM.

### Fedratinib and CC90009—GSEA in Primitive AMLs

For GSEA of bulk RNA-seq comparing Primitive Responders and Primitive Partial/Non-Responders to Fedratinib and CC90009, DESeq2 was used to perform differential expression analysis from raw counts with sequencing batch as a covariate. GSEA was subsequently performed using the DESeq2 test statistic as a rank metric querying the CGP and GO Biological Processes pathway databases (www.gsea-msigdb.org/gsea/msigdb/). For GSEA of single cell RNA-seq comparing NPM1 mutant LSPCs and other LSPCs, a rank list was generated using the Wilcoxon test through scanpy, and this was repeated for each LSPC subpopulation.

### Fedratinib and CC90009—Subclassification of Primitive AMLs

To impute NPM1 status among Primitive AML samples lacking targeted sequencing profiles, variance stabilized gene expression profiles of Primitive AMLs were filtered down to 39 genes differentially expressed in NPM1c mutant Primitive AMLs (FDR<0.01, log 2FC>1) compared to NPM1 wild-type Primitive AMLs, and subsequently used for dimensionality reduction with a local neighbourhood size of 5 and a minimum UMAP distance of 0.1, resulting in two Primitive AML clusters that completely separated NPM1 mutant from NPM1 wild-type samples. Samples with unknown NPM1 mutation status were subsequently assigned as NPM1 mutant or NPM1 wild-type according to their cluster membership.

### Fedratinib and CC90009 Combination Treatment

NOD.SCID mice were bred and housed in the University Health Network (UHN) animal care facility and all animal experiments were performed in accordance with guidelines approved by the UHN animal care committee. Ten-week-old NOD.SCID mice were irradiated (225 cGy) and pretreated with anti-CD122 antibody (200 μg per mouse), 24 hours prior to transplantation. Viably frozen mononucleated cells from AML patients were thawed, counted, and intrafemorally injected at the dose of 5 million cells per mouse. At day 21 post transplantation, treatment of either CC-90009 or Fedratinib alone with vehicle, or in combination, was initiated twice a day for 2 weeks. CC-90009 was given by intraperitoneal (IP) injections at the dose of 2.5 mg/kg and Fedratinib was dissolved in 0.5% methylcellulose and orally gavaged at 60 mg/kg. Following treatment, levels of AML engraftment were assessed to determine the efficacy of drug treatment against the disease in the mice. Cells collected from the injected right femur, non-injected bone marrow of each individual mouse were stained with human specific antibodies and evaluated by flow cytometry. Antibodies used for assessment of human AML engraftment include: CD45-APC, CD33-PE-Cy5, CD19-V450, CD34-APC-Cy7, CD15-FITC (BD), CD33-PE-Cy5, and CD14-PE (Beckman Coulter).

### Results and Discussion

### Single-Cell Characterization of Leukemia Stem and Progenitor Populations

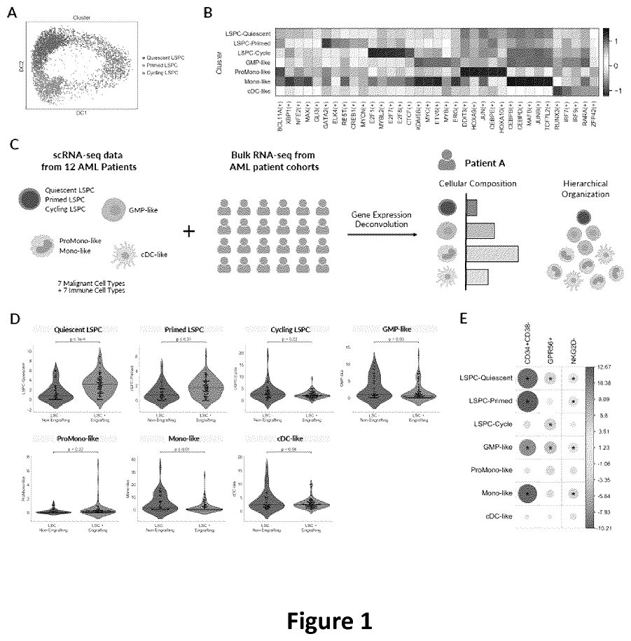

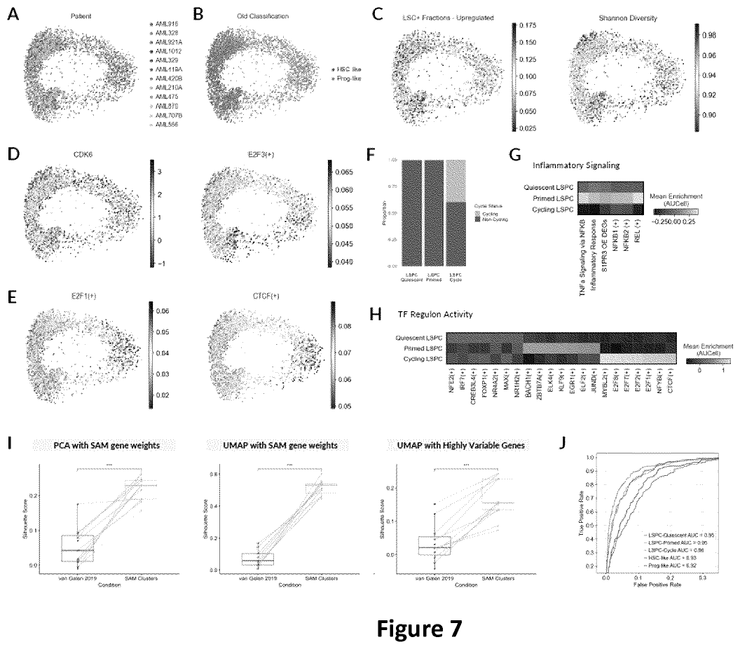

As a first step to uncover the organization of cellular hierarchies in AML, we re-analyzed the scRNA-seq data of 13,653 cells from 12 AML patients at diagnosis³² with a focus on primitive stem and progenitor blast populations (henceforth Leukemia Stem and Progenitor Cells, LSPCs). Using Self-Assembling Manifolds (SAM), an unsupervised approach to prioritize biologically relevant features among relatively homogenous cells³⁴, we previously identified two transcriptomic populations of normal human HSC: a deeply quiescent population with low transcriptome diversity (Non-Primed) and another residing in a shallower state of quiescence with higher CDK6 expression (Cycle-Primed)³⁵. We applied SAM to analyze LSPCs and identified three distinct LSPC populations shared across the 12 patients (FIG. 1A, FIG. 7A). One population had low transcriptome diversity and was enriched for core LSC programs but appeared otherwise inactive (FIG. 7C). We named this population Quiescent LSPC. The second population was enriched for CDK6 expression and targets of the cell cycle regulator E2F3 suggestive of cell cycle priming (FIG. 7D), as well as inflammatory signatures suggestive of priming for myeloid differentiation³⁶ (FIG. 7G). We named this population Primed LSPC. The third population exhibited enrichment for CTCF targets suggestive of stem cell activation³⁷ and broad enrichment of E2F targets indicating cell cycle progression, with 40% of cells classified as cycling (FIG. 7E-F, H). We named this third population Cycling LSPC. The existence of distinct cellular states provides a molecular basis for the known functional heterogeneity that is found within the LSC compartment 38. These new classes of Quiescent, Primed, and Cycling LSPC led to improved clustering performance over the prior ‘HSC-like’ and ‘Progenitor-like’ classification from van Galen et al³² (FIG. 7B, I-J). These new LSPC classes, together with the existing classification of more committed blasts by van Galen et al (GMP-like blasts resembling Granulocyte-Monocyte Progenitors, ProMono-like blasts resembling promonocytes, Mono-like blasts resembling monocytes, and cDC-like blasts resembling conventional Dendritic cells), provide a map of common leukemic blast states shared across these 12 AML patients, with each leukemic state having distinct molecular properties (FIG. 1B).

### Deconvolution of Constituent Cell Populations in AML

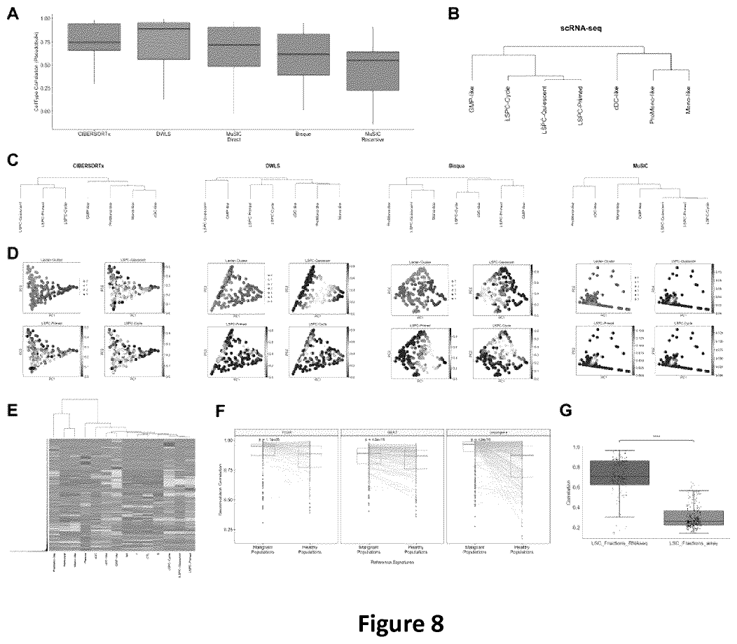

We next sought to understand how these defined AML cell populations and the hierarchies into which they are organized relate to functional, biological, and clinical properties of AML. High per-patient costs limit the scale of scRNA-seq analysis to small numbers of samples, restricting the ability to establish links between cellular states and clinical outcomes. As an alternative approach, we sought to employ gene expression deconvolution on bulk AML transcriptomes from publicly-available AML datasets. The transcriptome of any sample represents a mixture of RNA from every cell within the sample and thus its cellular composition can be inferred using known gene expression profiles of each component cell type^{39,40}. For deconvolution of bulk RNA-seq data from AML samples, we used single cell transcriptomes from seven malignant cell types described in FIG. 1B and seven non-leukemic immune cell types (Natural Killer, Naive T, CD8+ T, B, Plasma, Monocytes and cDCs) as a reference (FIG. 1C). To determine the best approach for our study, we compared 4 tools designed specifically for deconvolution of RNA-seq data using scRNA-seq reference profiles.⁴⁰⁻⁴³ CIBERSORTx performed best in estimating cell type composition from pseudo-bulk samples (FIG. 8A) and preserving relationships between malignant cell populations in deconvoluted bulk RNA-seq data (FIG. 8B-D). Deconvolution of AML patient cohorts performed significantly better when malignant cell populations were used as a reference rather than healthy hematopoietic counterparts, highlighting the importance of using AML-specific signatures (FIG. 8F). In addition, deconvolution performance was substantially higher for RNA-seq compared to microarray data (FIG. 8G). We thus employed CIBERSORTx for all deconvolution and restricted our analysis exclusively to RNA-seq data.

Quiescent LSPC Abundance Associates with Functional LSC Activity

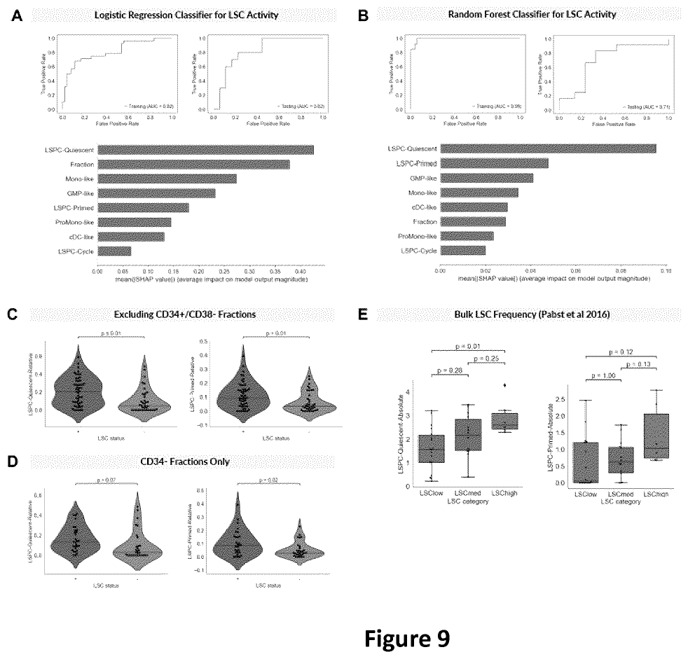

We first sought to determine whether any of our newly-defined LSPC cellular states were associated with LSC activity. The LSC state is functionally defined by whether a leukemic cell can initiate leukemia in vivo⁴⁴. We thus performed RNA-seq on 110 AML fractions that were previously evaluated for LSC activity through xenotransplantation²⁶ and applied deconvolution to determine the cell type composition of each fraction. Quiescent LSPC and Primed LSPC were both strongly enriched in LSC-positive fractions (qLSPC: p<1e-4; pLSPC: p<0.01) (FIG. 1D). Conversely, Mono-like blasts were enriched in LSC-negative fractions (p<0.01) along with Cycling LSPC (p=0.02), GMP-like (p=0.03), and cDC-like blasts (p=0.06) (FIG. 1D). Quiescent LSPC were consistently enriched in AML fractions sorted with LSC-associated markers (CD34+/CD38−²⁶, p=1e-5; GPR56+⁴⁵, p=0.01; NKG2D−⁴⁶, p=0.04) (FIG. 1E). Given that immunophenotype does not consistently predict LSC activity^{23,26,47}, we compared deconvolution against immunophenotype by training logistic regression and random forest classifiers to predict LSC activity in AML fractions based on cell type composition and CD34/CD38 status. In both models, Quiescent LSPC abundance was the most important predictor of LSC activity in AML fractions, outperforming immunophenotype (FIG. 9A-B). Quiescent LSPCs also remained enriched in LSC-positive fractions after excluding CD34+/CD38-fractions from the analysis (FIG. 9C-D). Finally, Quiescent LSPC enrichment was associated with high LSC frequency in samples from the Leucegene cohort assessed by limiting dilution analysis of bulk cells⁴⁵ (FIG. 9E). Collectively, these findings establish a new link between transcriptomic LSPC states and functionally-defined LSC at the apex of the hierarchy, suggesting that LSC activity can be inferred through deconvolution of patient hierarchies.

AML Hierarchy Composition Associates with Genetic Alterations and Clinical Outcomes

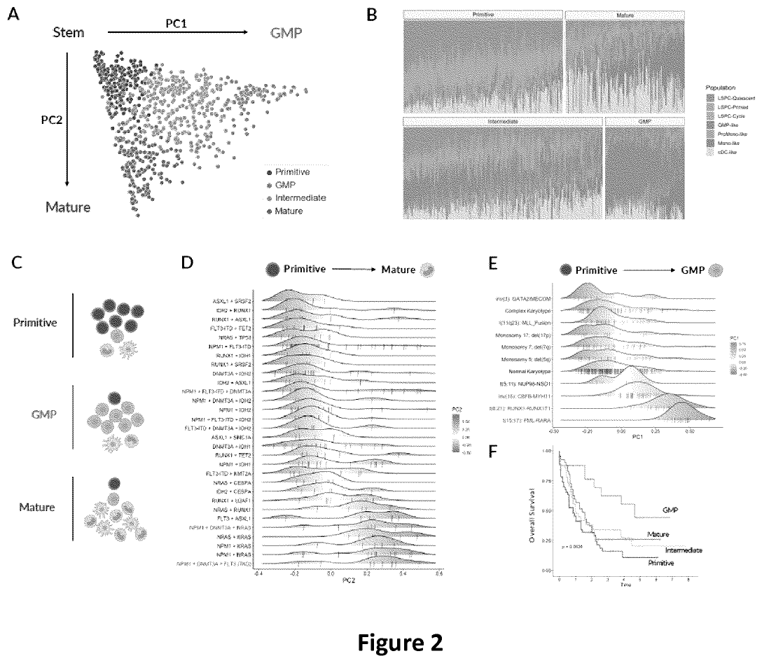

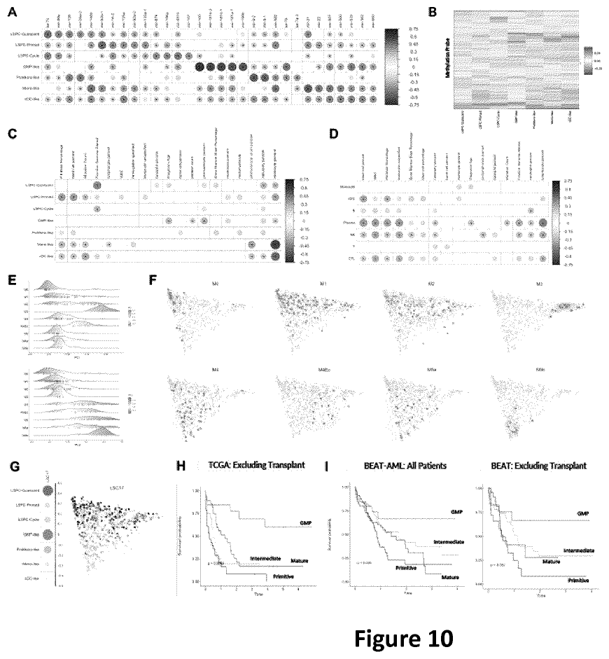

The differentiation properties of the LSCs sustaining each patient's AML can be captured by examining the full composition of the hierarchies that they generate. To examine how these hierarchies vary across patient samples and how they relate to molecular and clinical features of AML, we applied our deconvolution approach to infer the abundance of 7 leukemic cell types as well as 7 non-leukemic immune populations (described above) within 812 diagnostic patient samples from the TCGA⁴⁸, BEAT-AML⁴⁹, and Leucegene cohorts⁵⁰. Clustering patients based on the composition of their leukemic cells revealed four clusters of AML patients with distinct hierarchy compositions: Primitive (shallow hierarchy, LSPC-enriched), Mature (steep hierarchy, enriched for mature Mono-like and cDC-like blasts), GMP (dominated by GMP-like blasts), and Intermediate (balanced distribution) (FIG. 2A-C). Hierarchy composition was associated with multiple biological and clinical parameters including miRNA expression (FIG. 10A), global methylation profiles (FIG. 10B), age at diagnosis (Fig. S4C), WBC differential counts (FIG. 10C-E), and FAB class (FIG. 10E-F). We focused on cytogenetic and mutational correlates to understand the cellular states and hierarchies generated by common genetic drivers of AML.

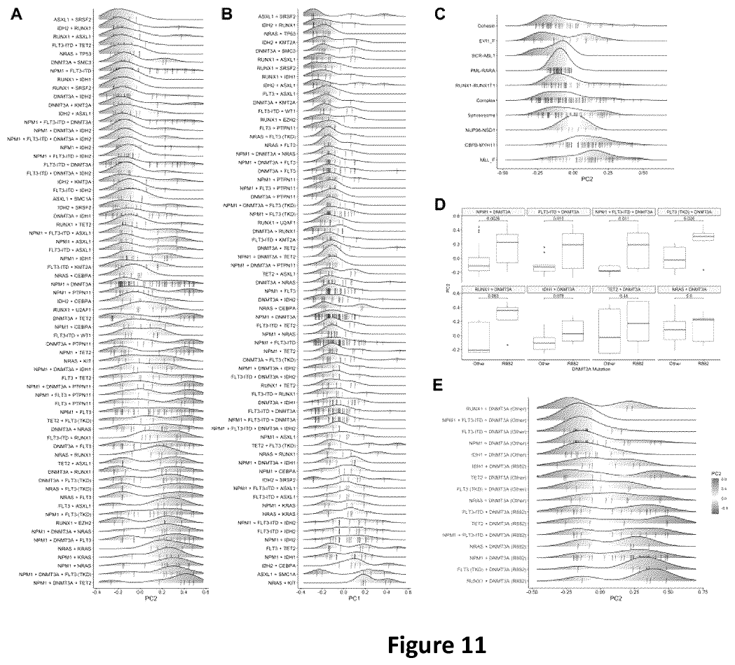

Patient hierarchies were separated along two principal components: PC1, spanning a continuum from Primitive to GMP (35% of variance) and PC2, spanning Primitive to Mature (28% of variance) (FIG. 2A). Cellular hierarchies generated by genetic mutations and their combinations primarily separated along PC2 (Primitive vs Mature) (FIG. 2D; FIG. 11A-B). Mutation combinations from Papaemmanuil et al⁵ that confer adverse risk generated shallow, Primitive hierarchies, while those conferring favourable risk generated committed, Mature hierarchies (FIG. 2D). Different mutations in the same gene could have different consequences on the resulting hierarchies. For example, DNMT3A R882 mutations were associated with more mature disease than other DNMT3A mutations (FIG. 11D-E). Hierarchies generated by cytogenetic alterations primarily separated along PC1 (Primitive vs GMP) (FIG. 2E; FIG. 11C), with adverse cytogenetic alterations generating Primitive hierarchies and favourable cytogenetic alterations generating GMP-dominant hierarchies (FIG. 2E).

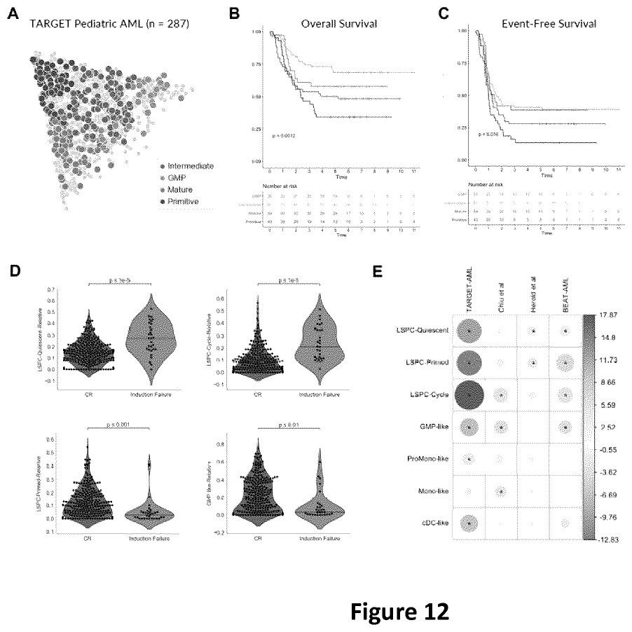

In addition to genetic biomarkers, stemness-based biomarkers were also linked to hierarchy composition. Notably, a high LSC17 score, which predicts a poor outcome following chemotherapy²⁶, was highest among patients with Primitive hierarchies and was correlated with Quiescent LSPC abundance (r=0.30, p=3e-19) and anti-correlated with GMP-like abundance (r=−0.28, p=5e-17) across the TCGA, BEAT-AML, and Leucegene cohorts (FIG. 10G). Indeed, Primitive hierarchies were directly associated with poor outcomes in TCGA and BEAT-AML cohorts, and this was even more pronounced after excluding patients who underwent bone marrow transplantation (FIG. 2F; FIG. 10H-I). Despite the fact that there is little overlap in genetic drivers between adult and pediatric AML⁵¹⁻⁵³, Primitive hierarchies were also associated with the worst outcomes in pediatric AML (FIG. 12A-C), pointing to the convergence of diverse genetic mechanisms in generating similar disease states. Moreover, both pediatric and adult patients who did not achieve complete remission following induction chemotherapy^{49,51,54,55} had higher Quiescent and Cycling LSPC abundance compared to patients who achieved remission (FIG. 12D-E). Together, these results indicate that variation in hierarchy composition is linked to intrinsic differences in chemotherapy response, suggesting that the prognostic value of genomic and stemness-based biomarkers in AML may in part be reflected by the cellular composition of the hierarchies with which they are associated.

### Shift Towards Primitive Hierarchies at Relapse

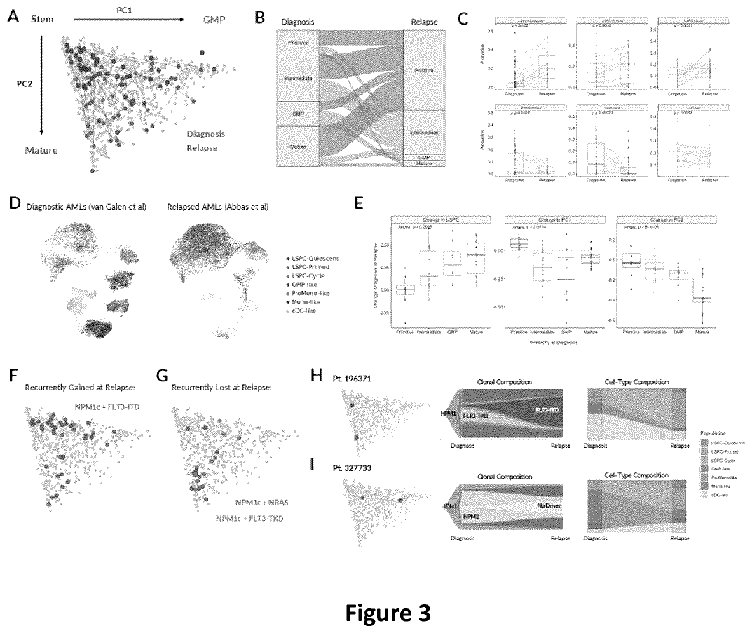

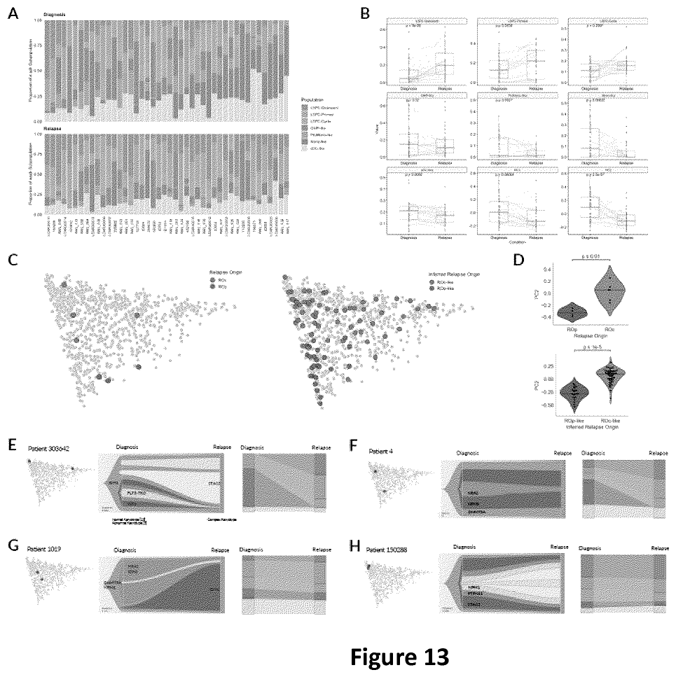

Given the associations we observed between AML hierarchy composition and chemotherapy response, we asked whether the composition of these hierarchies evolve over the course of disease. To answer this, we deconvoluted 44 pairs of samples collected at diagnosis and relapse following induction chemotherapy from four independent cohorts^{21,56-58} (FIG. 3A). At diagnosis, patient samples exhibited diverse hierarchy compositions, yet by relapse most samples were classified as Primitive (FIG. 3B). Indeed, 39 out of the 44 (89%) paired samples exhibited higher LSPC abundance at relapse, with relapse samples being particularly enriched for Quiescent LSPCs (p=9e-6) (FIG. 3C, FIG. 13A-B). To validate this finding at the single cell level, we analyzed scRNA-seq data from 8 relapsed AML patients and observed uniformly higher LSPC abundance as compared to 12 diagnostic AML samples from vanGalen et al³² (FIG. 3D). This consistent expansion of the stem cell compartment observed at relapse is corroborated by functional studies reporting a universal increase in LSC frequency from diagnosis to relapse^{21,59}.

Although the direction of shift in cellular composition from diagnosis to relapse was consistent across samples, the magnitude of each shift depended on the hierarchy classification at diagnosis. AMLs with Intermediate, GMP, or Mature hierarchies at diagnosis experienced extensive changes in cell type composition concomitant with expansion of the stem cell compartment to re-emerge as Primitive at relapse (FIG. 3E). In contrast, AMLs with Primitive hierarchies at diagnosis experienced minimal change in cell type composition from diagnosis to relapse (FIG. 3E), suggesting that some patients with Primitive hierarchies may already be further along the disease evolution trajectory by the time of diagnosis. These findings also provide further insight into two previously characterized origins of relapse in AML²¹. Our re-analysis showed that Relapse Origin-Primitive (ROp) cases exhibited Mature hierarchies at diagnosis that became replaced by self-renewing LSPCs at relapse. In contrast, Relapse Origin-Committed (ROc) cases already exhibited shallow, LSPC-dominant hierarchies at diagnosis and maintained their Primitive hierarchy at relapse (FIG. 13C-D).

Next, we asked whether these changes in cellular composition can be linked to discrete patterns of clonal evolution in AML. For example, FLT3-ITD alterations are recurrently gained at relapse while NRAS and FLT3-TKD alterations are recurrently lost at relapse in NPM1-mutant AMLs⁶⁰. Indeed, FLT3-ITD with NPM1c generated Primitive hierarchies (FIG. 3F), while NRAS or FLT3-TKD with NPM1c generated Mature hierarchies (FIG. 3G). For a subset of the patients we analyzed, changes in composition from diagnosis to relapse were concordant with patterns of clonal evolution (FIG. 3H, FIG. 13E-H). In other cases, shifts in composition occurred in the absence of clear genetic changes, suggestive of non-genetic modes of evolution (FIG. 3I). Irrespective of the evolutionary path taken, our findings show that relapse in AML involves a shift towards Primitive LSPC-dominant hierarchies, establishing stemness as a convergent property acquired across diverse pathways of AML evolution.

### The Primitive Vs Mature Axis Predicts Sensitivity to Targeted Therapies

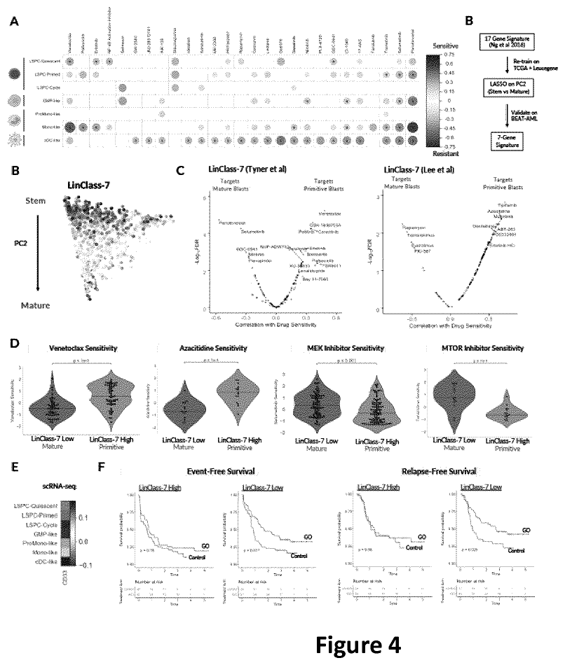

Having shown that outcomes following standard chemotherapy are tied to hierarchy composition, we investigated whether this association extended to molecularly targeted therapies. Specifically, we asked whether AML samples with different cellular compositions are vulnerable to different drugs. To this end, we integrated ex vivo drug sensitivity data from BEAT-AML with cell type abundance to generate drug sensitivity profiles for each malignant cell type (FIG. 4A). These revealed strong differences in drug responses between primitive blasts and mature blasts. Separation of drug responses occurred primarily along PC2 (Primitive vs Mature) with PC2 significantly correlated with response to 35 drugs (FDR<0.05). By contrast, PC1 (Primitive vs GMP) was not significantly correlated with sensitivity to any drug in the screen. These results point to cell type composition as a critical determinant of targeted therapy response.

To translate this finding to the clinic, we reasoned that a gene expression score approximating the Primitive vs Mature axis may offer a tool to infer blast sensitivity to a wide range of targeted therapies, enabling patients to be directed to the therapies from which they will most likely benefit. As a proof of concept, we turned to the LSC17 score, a stemness-based gene expression score for rapid risk stratification in AML currently being evaluated in clinical trials^{26,27} Given that the LSC17 score was associated with leukemic hierarchy composition (FIG. 10G), we reasoned that deriving a sub-score from these 17 genes to estimate PC2 may provide a rapidly deployable tool to inform therapy selection using data from the existing LSC17 assay. We thus retrained the LSC17 genes on PC2 using LASSO with TCGA and Leucegene as a training set and BEAT-AML as a validation set. We identified a 7-gene lineage classification sub-score (hereafter LinClass-7) which was strongly anti-correlated with PC2 (TCGA: r=−0.83, Leucegene: r=−0.81, BEAT: r=−0.82), such that higher scores associated with Quiescent and Primed LSPCs and lower scores associated with Mono and cDC-like blasts (FIG. 4B, 14A-C). Correlation of the LinClass-7 score with drug sensitivity from drug screens performed by Tyner et al⁴⁹ and Lee et al⁶¹ identified 45 drugs targeting either primitive blasts (e.g. Venetoclax, Azacytidine, Palbociclib, and Mubritinib, FIG. 4C) or mature blasts (e.g. MEK/MTOR inhibitors, FIG. 4D).

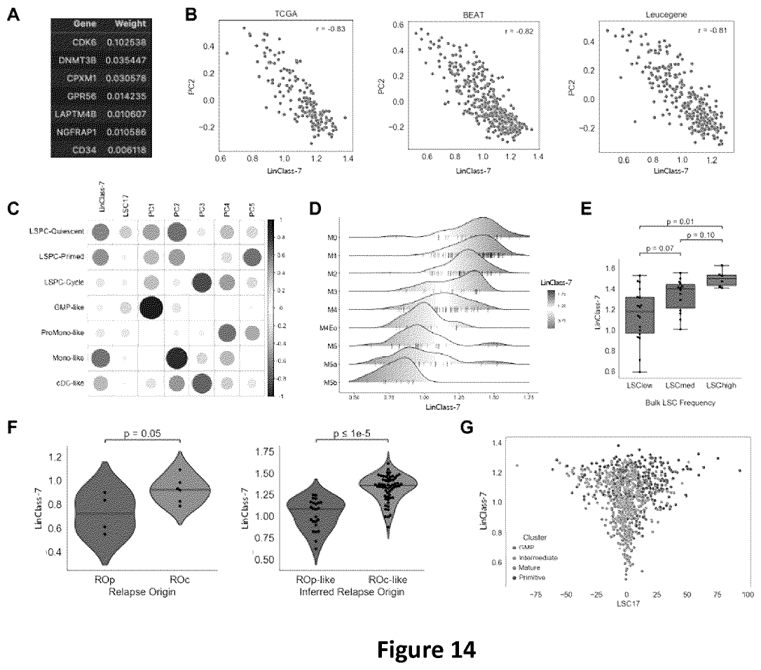

To examine the clinical relevance of the LinClass-7 score we performed subgroup analysis of the ALFA-0701 trial⁶², which evaluated low fractionated doses of Gemtuzumab Ozogamicin (GO), a drug-conjugated antibody targeting CD33, in combination with standard chemotherapy. CD33 is highly expressed on mature myeloid blasts but has lower expression among LSPCs (FIG. 4E). Addition of GO to standard chemotherapy was associated with significantly longer event-free and relapse-free survival for patients with LinClass-7 low (mature) AML, although this association did not extend to overall survival. In contrast, patients with LinClass-7 high primitive AML derived no significant survival benefit from GO (FIG. 4F). LinClass-7 also had a range of other biological correlates: it was highly associated with FAB class (FIG. 14D), bulk LSC frequency (FIG. 14E) and relapse origin²¹ (FIG. 14F). Importantly, the LinClass-7 and LSC17 scores are orthogonal (FIG. 14G), providing complementary information to loosely reconstruct the map of cellular hierarchies in AML.

Taken together, these findings demonstrate that the differentiation state as reflected by hierarchy composition governs sensitivity to a wide array of investigational therapies. By distilling the Primitive vs Mature axis into the LinClass-7 score that can be measured through the clinically-deployed LSC17 NanoString assay, we provide a novel approach for hierarchy-based prediction of sensitivity to a range of therapies with the potential to guide therapy selection.

### Changes in Cellular Composition in Response to Drug Treatment

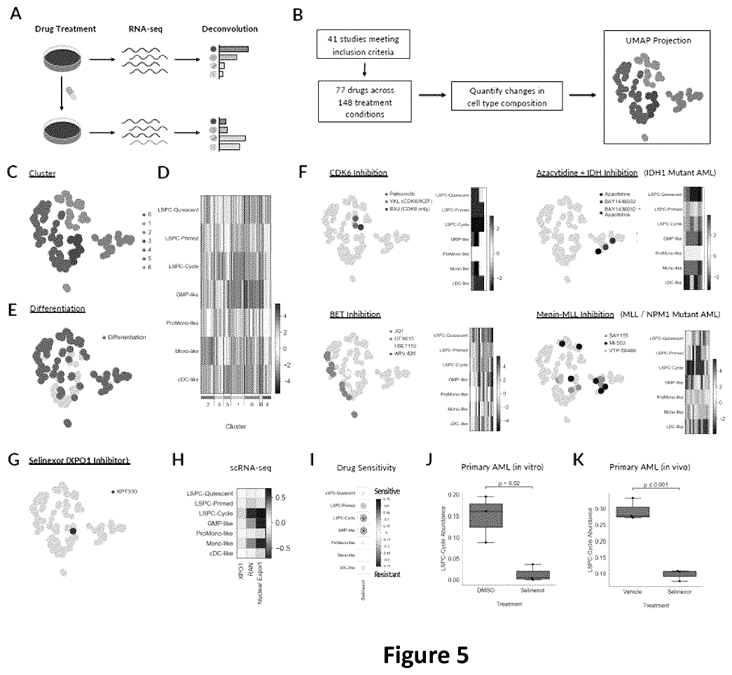

We next sought to determine how our leukemic hierarchy framework can be deployed in the context of drug development. Drug candidates are often identified based on reduction in viability of bulk leukemia cells or cell lines, yet this measure lacks critical information pertaining to the subpopulations of cells that are targeted or that persist after treatment. To understand how drug treatment affects cellular composition, we deconvoluted RNA-seq data from 41 datasets in GEO and ArrayExpress with human AML cells sequenced before and after drug treatment (FIG. 5A-B).

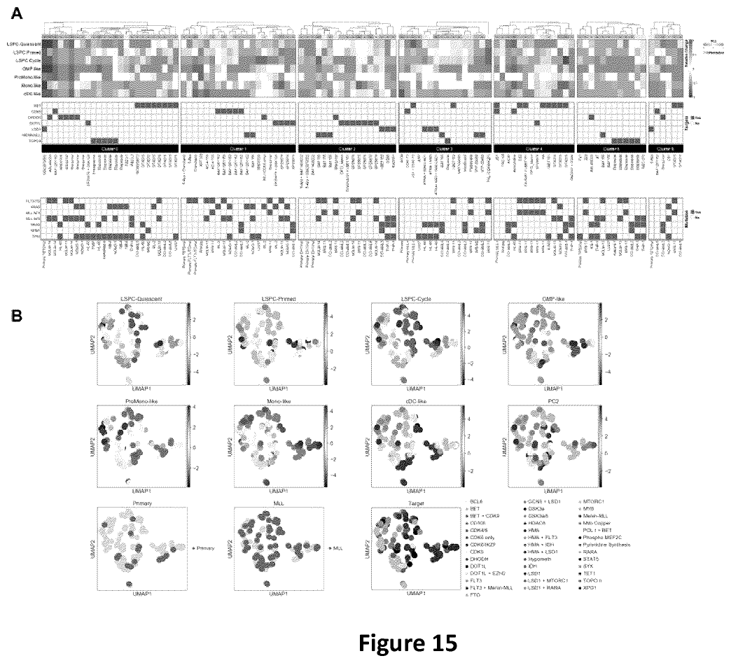

We visualized the changes in cellular composition following drug treatment in low-dimensional UMAP space and clustered drugs based on the cell composition changes they induced (FIG. 5C-E, 15B). Across 153 treatment conditions, 125 resulted in significant changes in cell-type composition. We observed heterogeneous patterns of change across drug families, some corresponding to known mechanisms of sensitivity or resistance. For example, depletion of Cycling LSPCs by CDK6 inhibitors was accompanied by an increase in non-cycling Quiescent LSPC and Primed LSPC populations (FIG. 5F), consistent with the role of CDK6 in quiescence exit⁶³. A combination of azacytidine with an IDH1 inhibitor, which blocks LSC self-renewal and induce myeloid differentiation in IDH1-mutant AML⁶⁴, showed reduced LSPC abundance (particularly Quiescent LSPC) in deconvoluted primary samples (FIG. 5F). Seventy-seven treatment conditions led to a significant increase in PC2, indicative of differentiation, yet most of these treatments resulted in depletion of GMP-like blasts (69%), with fewer treatments depleting the more primitive qLSPC (30%) or pLSPC populations (14%) (FIG. 15A). For example, ATRA induced differentiation predominantly from GMP-like blasts (FIG. 5F). In contrast, differentiation induced by the DHODH inhibitor Brequinar was accompanied by a reduction in Quiescent LSPC abundance, suggesting that this drug may better deplete the stem cell compartment (FIG. 5F).

In some cases, the cell population depleted by a drug corresponded to the expression of the drug target. For example, we analyzed the cellular response to Selinexor, a drug targeting the nuclear export protein XPO1 (FIG. 5G). XPO1 and nuclear export processes were enriched in the Cycling LSPC population at the single cell level (FIG. 5H), and depletion of this cell population was correlated with ex vivo Selinexor sensitivity in the BEAT-AML screen (FIG. 5I). To support this prediction with independent functional evidence, treatment of primary AML samples with Selinexor resulted in depletion of the Cycling LSPC population both in vitro⁶⁵ and in vivo⁶⁶ (FIG. 5J-K) across diverse genetic backgrounds.

Taken together, our data shed light on the changes in cellular composition that follow drug treatment and offer a functionally relevant read-out for prioritizing candidate drugs in preclinical settings.

Hierarchy-Based Stratification Predicts In Vivo Drug Response and Identifies Drugs with Complementary Efficacy Profiles

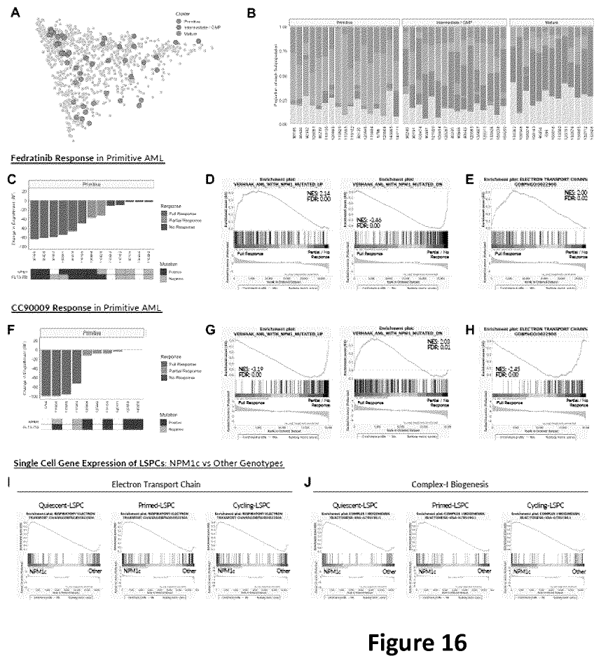

Having demonstrated an association of cell type composition with drug response, we asked whether a hierarchy-based classification system can predict drug response in vivo. To this end we turned to patient-derived xenograft (PDX) response data generated by our group for two drugs: fedratinib, a JAK2 inhibitor approved for treatment of myeloproliferative neoplasms, and CC-90009⁶⁷, an immunomodulatory (IMiD) agent that induces cereblon-mediated degradation of GSPT1⁶⁸. A total of 46 independent AML samples were treated in PDX models (n=32 for fedratinib and n=30 for CC90009) across 658 drug- or vehicle-treated xenografted mice. Deconvoluted RNA-seq profiles from the primary patient samples prior to xenotransplantation were clustered based on hierarchy composition and categorized as Primitive, Intermediate/GMP, or Mature (FIG. 16A-B).

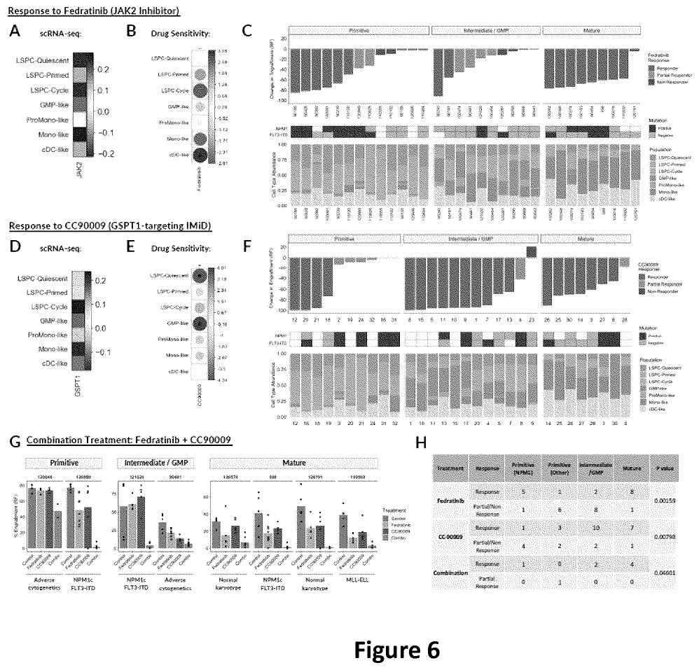

The primary target of Fedratinib is JAK2, which is predominantly expressed in Mono-like and cDC-like blasts at the single cell level (FIG. 6A). These mature blasts were enriched in patient samples that responded well to fedratinib in vivo (FIG. 6B). Subgroup analysis of fedratinib response showed high efficacy in AMLs with Mature hierarchies (88% response rate), while response rates among other hierarchy types were poor (46% for Primitive and 20% for Intermediate/GMP) (FIG. 6C). CC-90009 targets GSPT1, whose expression is enriched in Cycling LSPC and GMP-like blasts at the single cell level (FIG. 6D). GMP-like blasts were enriched in responders while Quiescent LSPCs were enriched in partial and non-responders (FIG. 6E). Subgroup analysis showed high CC-90009 efficacy in AMLs with Mature and Intermediate/GMP hierarchies, with 88% and 83% response rates, respectively. In contrast, those with Primitive hierarchies had heterogeneous responses at a rate of 40% (FIG. 6F).

To better understand the heterogeneous responses to fedratinib and CC-90009 among patient samples, we compared the molecular features of responding and non-responding AML samples for both Fedratinib and CC-90009 treatment conditions. Among Primitive AMLs, NPM1c mutations and concomitant electron transport chain (ETC) signatures were associated with favorable response to fedratinib and poor response to CC-90009, while Primitive AMLs lacking NPM1c mutation and ETC signatures demonstrated favorable response to CC-90009 and poor response to fedratinib (FIG. 6D,H, FIG. 16C-H). We confirmed at the single cell level that NPM1c-mutant LSPCs were transcriptionally enriched for ETC Complex I-related pathways (FIG. 161-J). Importantly, the association of NPM1c signatures with Fedratinib and CC-90009 response among Primitive AMLs did not extend to other hierarchy subtypes. Given the NPM1c-based response dichotomy to fedratinib and CC-90009 among Primitive hierarchies, as well as the sensitivity of Intermediate/GMP hierarchies to CC-90009 and Mature hierarchies to both drugs, we reasoned that a combination of the two drugs may show efficacy against a broader range of samples than either drug alone. To test this hypothesis, we treated PDXs from eight AML samples of diverse hierarchy types with a combination of fedratinib and CC-90009. Seven of the eight patient samples tested responded fully to combination treatment with virtual elimination of the leukemic graft, despite variable responses to single agent fedratinib or CC-90009 (FIG. 6G).

Overall, responses to Fedratinib, CC-90009, and combination treatment in PDX models were all significantly associated with hierarchy composition (FIG. 6H). These data establish that stratification of AML cases on the basis of hierarchy composition accompanied by mutational information enables prediction of those likely to benefit from specific therapies, and also facilitates the design of combination regimens through selection of drugs demonstrating complementarity in their efficacy profiles.

### Discussion

Here we introduce a new approach for understanding heterogeneity in AML based on the composition of each patient's leukemic hierarchy. Analysis of patient-specific variation in hierarchy composition across large cohorts captured and integrated information on genomic profiles, stem cell properties, and clinical outcomes; something that could not be achieved by applying the genomic or stem-cell models alone. For example, LSPC abundance inferred through deconvolution accurately reflected functional LSC activity. Despite the wide diversity of genetic drivers, we were able to distill hierarchy composition into four main classes. The fact that distinct drivers generate similar hierarchy compositions implies convergence in how mutations perturb LSC function and impair hematopoietic differentiation. In this way, hierarchy composition provides a means of understanding how different genetic subgroups relate to one another and, more broadly, how genetic alterations relate to LSC properties. Thus, understanding AML through the lens of cellular hierarchies enables a more comprehensive view of biological heterogeneity in AML.

Considering hierarchy composition improves our understanding of chemotherapy response in AML. Primitive hierarchies were directly associated with poor outcomes in adult and pediatric AML, and both genomic and stemness-based markers of adverse risk captured patients with these hierarchies, suggesting that differences in hierarchy composition may help to explain heterogeneous outcomes following induction chemotherapy. In line with these findings, relapse following chemotherapy was associated with a recurrent shift towards a Primitive hierarchy composition. Clonal evolution is one way to achieve this transition, wherein mutations that produce primitive phenotypes preferentially expand at relapse while those that generate mature phenotypes are lost. Epigenetic evolution has also been observed between diagnosis and relapse in AML, which can occur independently of genetic evolution⁵⁶. Whether through genetic or epigenetic evolution, progression in AML is accompanied by a loss of the hierarchical structure and differentiation programs inherited from normal development. Collectively, these findings link hierarchy composition to both response and evolution following chemotherapy.

Most importantly, analysis of leukemic hierarchy composition helps to address the emerging challenge of therapy selection. The molecular circuits underlying survival differ across leukemic cells at each stage of differentiation, and it follows that individual drugs may only be effective against a subset of leukemic cells from each patient. This is well established for chemotherapy and has recently been demonstrated for Venetoclax in combination with Azacytidine (Ven-Aza), wherein LSPCs expressing BCL-2 were effectively eliminated but resistance and relapse emerged from a pro-monocytic blast population expressing MCL-1^{69,70}. Our work extends this paradigm by showing that hierarchy composition underlies heterogeneous responses to a wide range of investigational therapies across ex vivo screens, in vivo PDX models, and within the ALFA-0701 trial. We also provided a proof-of-concept that hierarchy composition can be approximated through simple regression-based gene expression scores, which could be rapidly measured in the clinic to facilitate therapy selection. Our study points to alternative treatment approaches in which drug combinations could be designed to target each of the leukemic cell types present in the disease. Indeed, some drug pairs that were predicted to target distinct AML cell types by our hierarchy analysis have been shown to have synergistic activity by other groups^{70,71}. Thus, a treatment approach that considers hierarchy composition has the potential for broader applicability over approaches that target specific genetic mutations alone. Together, these findings set the foundation for a novel precision medicine framework for AML.

A major implication of our study is that while AML hierarchy composition is both prognostic (capturing survival outcomes following induction chemotherapy) and predictive (capturing response to biologically targeted therapies), these are captured by separate axes of variation: PC1 (Stem to GMP) is highly prognostic but not predictive of drug response, while PC2 (Stem to Mature) is highly predictive but not prognostic. Accordingly, the LSC17 prognostic score was most enriched among patients with Primitive hierarchies, and were lowest among patients with GMP-dominant hierarchies. Thus, our motivation in training LinClass-7 was to develop a companion score to LSC17 that could capture the predictive axis.

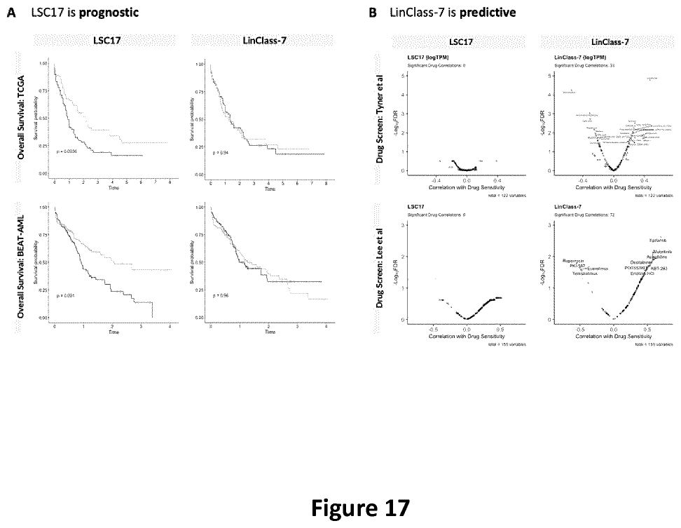

In this way, the LinClass-7 score is not meant to be prognostic. Indeed, there is no significant difference in overall survival outcomes between LinClass-7 High and LinClass-7 Low patients in either the TCGA or BEAT-AML cohorts. This is in contrast to LSC17, for which a median split gives rise to two groups of patients with stark differences in their survival outcomes (FIG. 17A). Instead, LinClass-7 is meant to be predictive, as shown through the 33 and 72 significant (FDR<0.05) correlations with ex vivo drug sensitivity from two primary AML drug screens, respectively. In contrast, the LSC17 score did not exhibit any significant correlations with ex vivo drug sensitivity from either drug screen (FIG. 17B).

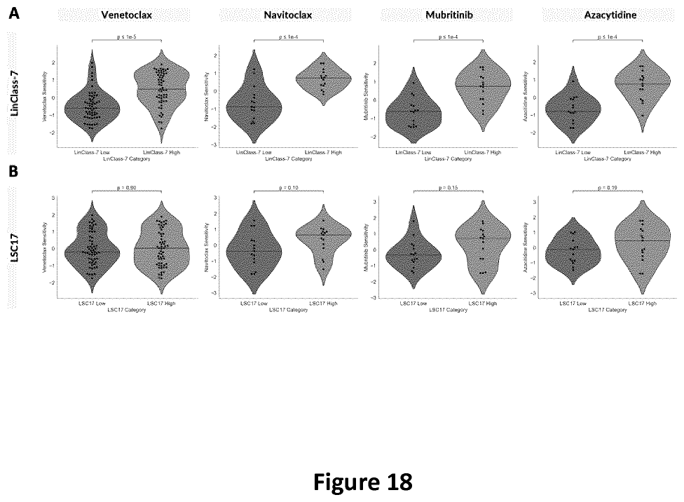

Importantly, many of the drugs with which LinClass-7 is strongly correlated are either directly clinically relevant or progressing through clinical trials. For example, patient samples split based on high or low scores of LinClass-7 show stark differences in sensitivity to BCL2 inhibitor Venetoclax, BCL2/BCL-XL inhibitor Navitoclax, oxidative phosphorylation inhibitor Mubritinib, and the RNA hypomethylating agent Azacytidine, among others (FIG. 18A). The combination of Venetoclax and Azacytidine has recently been adopted into the clinic, while Mubritinib was recently ‘re-discovered’ in 2019 (Bacelli et al Cancer Cell) and is currently in clinical trials. However, patient samples split based on LSC17 exhibited no significant difference in sensitivity to these drugs (FIG. 18B). Thus LinClass-7 captures an axis of variation in AML that is distinct from LSC17, serving a different purpose in predicting response to targeted agents rather than survival outcomes following chemotherapy.

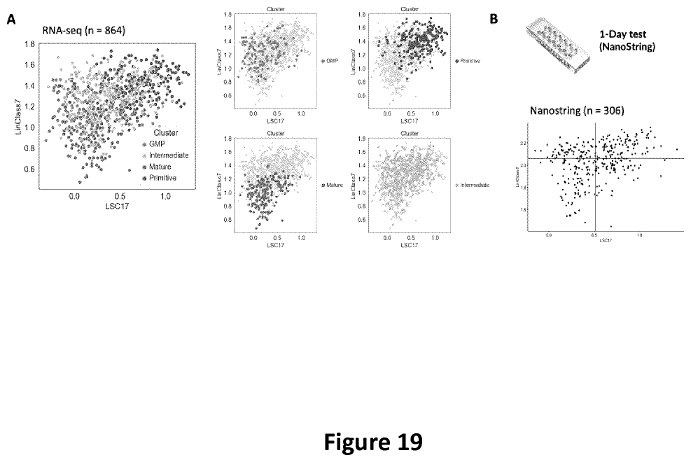

Plotting the distribution of patient samples by LSC17 and LinClass-7 loosely recapitulates the primary axes of hierarchy variation, separating Primitive, GMP, and Mature AMLs (FIG. 19A). Thus the LSC17 and LinClass-7 scores, measurable through the same 17-gene NanoString assay, allow for both prognostic and predictive stratification of patient samples while also providing salient information on patient hierarchy composition (FIG. 19B).

Our findings have immediate implications for pre-clinical studies of novel AML therapies, and our deconvolution workflow can be readily adopted by researchers and clinicians studying AML. However, further validation will be required to deploy this framework into the clinic. Hierarchy-based subgroup analysis of response data from recently completed or ongoing trials of novel AML therapies will be particularly important to validate the clinical utility of this approach. To build upon this framework, it will also be important to study the LSCs that sustain each type of hierarchy in order to develop therapies that can induce durable long-term remissions. Last, by providing a blueprint for translating insights from scRNA-seq studies into the clinic, we anticipate that this approach could also be applied to other cancers and have implications well beyond the AML field.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCES

Hungerford, D. A. & Nowell, P. C. A minute chromosome in human chronic granulocytic leukemia. Science 132, 1497-1499 (1960).
2. Bennett, J. M. et al. Proposals for the classification of the acute leukaemias. French-American-British (FAB) co-operative group. Br. J. Haematol. 33, 451-458 (1976).
3. Bennett, J. M. et al. Proposed revised criteria for the classification of acute myeloid leukemia. A report of the French-American-British Cooperative Group. Ann. Intern. Med. 103, 620-625 (1985).
4. Mrózek, K., Heerema, N. A. & Bloomfield, C. D. Cytogenetics in acute leukemia. Blood Rev. 18, 115-136 (2004).
5. Papaemmanuil, E. et al. Genomic Classification and Prognosis in Acute Myeloid Leukemia. N. Engl. J. Med. 374, 2209-2221 (2016).
6. Klco. J. M. et al. Functional heterogeneity of genetically defined subclones in acute myeloid leukemia. Cancer Cell 25, 379-392 (2014).
7. Till, J. E. & McCULLOCH, E. A. A direct measurement of the radiation sensitivity of normal mouse bone marrow cells. Radiat. Res. 14, 213-222 (1961).
8. Clarkson, B., Ohkita, T., Ota, K. & Fried, J. Studies of cellular proliferation in human leukemia. I. Estimation of growth rates of leukemic and normal hematopoietic cells in two adults with acute leukemia given single injections of tritiated thymidine. J. Clin. Invest. 46, 506-529 (1967).
9. Clarkson, B. D. The survival value of the dormant state in neoplastic and normal cell populations. Control of proliferation in animal cells 1, 945-972 (1974).
10. Minden, M. D., Till, J. E. & McCulloch, E. A. Proliferative state of blast cell progenitors in acute myeloblastic leukemia (AML). Blood 52, 592-600 (1978).
11. Griffin, J. D., Larcom, P. & Schlossman, S. F. Use of surface markers to identify a subset of acute myelomonocytic leukemia cells with progenitor cell properties. Blood 62, 1300-1303 (1983).
12. Wouters, R. & Lowenberg, B. On the maturation order of AML cells: a distinction on the basis of self-renewal properties and immunologic phenotypes. Blood 63, 684-689 (1984).
13. Sabbath, K. D., Ball, E. D., Larcom, P., Davis, R. B. & Griffin, J. D. Heterogeneity of clonogenic cells in acute myeloblastic leukemia. J. Clin. Invest. 75, 746-753 (1985).
14. Buick, R. N., Minden, M. D. & McCulloch, E. A. Self-renewal in culture of proliferative blast progenitor cells in acute myeloblastic leukemia. Blood 54, 95-104 (1979).
15. Chang, L. J., Till, J. E. & McCulloch, E. A. The cellular basis of self renewal in culture by human acute myeloblastic leukemia blast cell progenitors. J. Cell. Physiol. 102, 217-222 (1980).
16. Buick, R. N., Chang, L. J., Messner, H. A., Curtis, J. E. & McCulloch, E. A. Self-renewal capacity of leukemic blast progenitor cells. Cancer Res. 41, 4849-4852 (1981).
17. McCulloch, E. A. Stem cells in normal and leukemic hemopoiesis (Henry Stratton Lecture, 1982). Blood 62, 1-13 (1983).
18. Lapidot, T. et al. A cell initiating human acute myeloid leukaemia after transplantation into SCID mice. Nature 367, 645-648 (1994).
19. Bonnet. D. & Dick, J. E. Human acute myeloid leukemia is organized as a hierarchy that originates from a primitive hematopoietic cell. Nat. Med. 3, 730-737 (1997).
20. Terpstra. W. et al. Fluorouracil selectively spares acute myeloid leukemia cells with long-term growth abilities in immunodeficient mice and in culture. Blood 88, 1944-1950 (1996).
21. Shlush, L. I. et al. Tracing the origins of relapse in acute myeloid leukaemia to stem cells. Nature 547, 104-108 (2017).
22. Gentles, A. J., Plevritis, S. K., Majeti, R. & Alizadeh, A. A. Association of a leukemic stem cell gene expression signature with clinical outcomes in acute myeloid leukemia. JAMA 304, 2706-2715 (2010).
23. Eppert, K. et al. Stem cell gene expression programs influence clinical outcome in human leukemia. Nat. Med. 17, 1086-1093 (2011).
24. Levine, J. H. et al. Data-Driven Phenotypic Dissection of AML Reveals Progenitor-like Cells that Correlate with Prognosis. Cell 162, 184-197 (2015).
25. Jung, N., Dai, B., Gentles, A. J., Majeti, R. & Feinberg, A. P. An LSC epigenetic signature is largely mutation independent and implicates the HOXA cluster in AML pathogenesis. Nat. Commun. 6, 8489 (2015).
26. Ng. S. W. K. et al. A 17-gene stemness score for rapid determination of risk in acute leukaemia. Nature 540, 433-437 (2016).
27. Murphy. T. et al. Trial in Progress: Feasibility and Validation Study of the LSC17 Score in Acute Myeloid Leukemia Patients. Blood 134, 2682-2682 (2019).
28. Elsayed, A. H. et al. A six-gene leukemic stem cell score identifies high risk pediatric acute myeloid leukemia. Leukemia 34, 735-745 (2020).
29. Pierce, G. B. Neoplasms, differentiations and mutations. Am. J. Pathol. 77, 103-118 (1974).
30. Pierce, G. B. & Speers, W. C. Tumors as caricatures of the process of tissue renewal: prospects for therapy by directing differentiation. Cancer Res. 48, 1996-2004 (1988).
31. Kreso, A. & Dick, J. E. Evolution of the cancer stem cell model. Cell Stem Cell 14, 275-291 (2014).
32. van Galen, P. et al. Single-Cell RNA-Seq Reveals AML Hierarchies Relevant to Disease Progression and Immunity. Cell 176, 1265-1281.e24 (2019).
33. Wu, J. et al. A single-cell survey of cellular hierarchy in acute myeloid leukemia. J. Hematol. Oncol. 13, 128 (2020).
34. Tarashansky, A. J., Xue, Y., Li, P., Quake, S. R. & Wang, B. Self-assembling manifolds in single-cell RNA sequencing data. Elife 8. (2019).
35. Xie, S. Z. et al. Sphingolipid Modulation Activates Proteostasis Programs to Govern Human Hematopoietic Stem Cell Self-Renewal. Cell Stem Cell (2019) doi: 10.1016/j.stem.2019.09.008.
36. Xie, S. Z. et al. Sphingosine-1-phosphate receptor 3 potentiates inflammatory programs in normal and leukemia stem cells to promote differentiation. Blood Cancer Discov 2, 32-53 (2021).
37. Takayama, N. et al. The Transition from Quiescent to Activated States in Human Hematopoietic Stem Cells Is Governed by Dynamic 3D Genome Reorganization. Cell Stem Cell 28, 488-501.e10 (2021).
38. Hope, K. J., Jin, L. & Dick, J. E. Acute myeloid leukemia originates from a hierarchy of leukemic stem cell classes that differ in self-renewal capacity. Nat. Immunol. 5, 738-743 (2004).
39. Newman, A. M. et al. Robust enumeration of cell subsets from tissue expression profiles. Nat. Methods 12, 453-457 (2015).
40. Newman, A. M. et al. Determining cell type abundance and expression from bulk tissues with digital cytometry. Nat. Biotechnol. 37, 773-782 (2019).
41. Tsoucas, D. et al. Accurate estimation of cell-type composition from gene expression data. Nat. Commun. 10, 2975 (2019).
42. Jew. B. et al. Accurate estimation of cell composition in bulk expression through robust integration of single-cell information. Nat. Commun. 11, 1971 (2020).
43. Wang, X., Park, J., Susztak, K., Zhang, N. R. & Li. M. Bulk tissue cell type deconvolution with multi-subject single-cell expression reference. Nat. Commun. 10, 380 (2019).
44. Dick. J. E. Stem cell concepts renew cancer research. Blood 112, 4793-4807 (2008).
45. Pabst, C. et al. GPR56 identifies primary human acute myeloid leukemia cells with high repopulating potential in vivo. Blood 127, 2018-2027 (2016).
46. Paczulla, A. M. et al. Absence of NKG2D ligands defines leukaemia stem cells and mediates their immune evasion. Nature 572, 254-259 (2019).
47. Quek, L. et al. Genetically distinct leukemic stem cells in human CD34-acute myeloid leukemia are arrested at a hemopoietic precursor-like stage. J. Exp. Med. 213, 1513-1535 (2016).
48. Cancer Genome Atlas Research Network et al. Genomic and epigenomic landscapes of adult de novo acute myeloid leukemia. N. Engl. J. Med. 368, 2059-2074 (2013).
49. Tyner, J. W. et al. Functional genomic landscape of acute myeloid leukaemia. Nature 562, 526-531 (2018).
50. Marquis, M. et al. High expression of HMGA2 independently predicts poor clinical outcomes in acute myeloid leukemia. Blood Cancer J. 8, 68 (2018).
51. Bolouri, H. et al. The molecular landscape of pediatric acute myeloid leukemia reveals recurrent structural alterations and age-specific mutational interactions. Nat. Med. 24, 103-112 (2018).
52. Marceau-Renaut, A. et al. Molecular Profiling Defines Distinct Prognostic Subgroups in Childhood AML: A Report From the French ELAM02 Study Group. Hemasphere 2, e31 (2018).
53. Duployez, N. et al. The stem cell-associated gene expression signature allows risk stratification in pediatric acute myeloid leukemia. Leukemia 33, 348-357 (2019).
54. Chiu, Y.-C. et al. Integrating resistance functions to predict response to induction chemotherapy in de novo acute myeloid leukemia. Eur. J. Haematol. 103, 417-425 (2019).
55. Herold, T. et al. A 29-gene and cytogenetic score for the prediction of resistance to induction treatment in acute myeloid leukemia. Haematologica 103, 456-465 (2018).
56.
Li, S. et al. Distinct evolution and dynamics of epigenetic and genetic heterogeneity in acute myeloid leukemia. Nat. Med. 22, 792-799 (2016).
57. Cocciardi, S. et al. Clonal evolution patterns in acute myeloid leukemia with NPM1 mutation. Nat. Commun. 10, 2031 (2019).
58. Christopher. M. J. et al. Immune Escape of Relapsed AML Cells after Allogeneic Transplantation. N. Engl. J. Med. 379, 2330-2341 (2018).
59. Ho. T.-C. et al. Evolution of acute myelogenous leukemia stem cell properties after treatment and progression. Blood 128, 1671-1678 (2016).
60. Vosberg, S. & Greif, P. A. Clonal evolution of acute myeloid leukemia from diagnosis to relapse. Genes Chromosomes Cancer 58, 839-849 (2019).
61. Lee, S.-I. et al. A machine learning approach to integrate big data for precision medicine in acute myeloid leukemia. Nat. Commun. 9, 42 (2018).
62. Castaigne, S. et al. Effect of gemtuzumab ozogamicin on survival of adult patients with de-novo acute myeloid leukaemia (ALFA-0701): a randomised, open-label, phase 3 study. Lancet 379, 1508-1516 (2012).
63. Laurenti, E. et al. CDK6 levels regulate quiescence exit in human hematopoietic stem cells. Cell Stem Cell 16, 302-313 (2015).
64. Chaturvedi, A. et al. Synergistic activity of IDH1 inhibitor BAY1436032 with azacitidine in IDH1 mutant acute myeloid leukemia. Haematologica 106, 565-573 (2021).
65. Brunetti, L. et al. Mutant NPM1 Maintains the Leukemic State through HOX Expression. Cancer Cell 34, 499-512.e9 (2018).
66. Etchin, J. et al. Activity of a selective inhibitor of nuclear export, selinexor (KPT-330), against AML-initiating cells engrafted into immunosuppressed NSG mice. Leukemia 30, 190-199 (2016).
67. Chen, W. C. et al. An Integrated Analysis of Heterogeneous Drug Responses in Acute Myeloid Leukemia That Enables the Discovery of Predictive Biomarkers. Cancer Res. 76, 1214-1224 (2016).
68. Surka. C. et al. CC-90009, a novel cereblon E3 ligase modulator, targets acute myeloid leukemia blasts and leukemia stem cells. Blood 137, 661-677 (2021).
69. Pei, S. et al. Monocytic Subclones Confer Resistance to Venetoclax-Based Therapy in Patients with Acute Myeloid Leukemia. Cancer Discov. (2020) doi: 10.1158/2159-8290.CD-19-0710.
70. Kuusanmaki, H. et al. Phenotype-based drug screening reveals association between venetoclax response and differentiation stage in acute myeloid leukemia. Haematologica 105, 708-720 (2020).
71. Han. L. et al. Concomitant targeting of BCL2 with venetoclax and MAPK signaling with cobimetinib in acute myeloid leukemia models. Haematologica 105, 697-707 (2020).
72. Lun, A. T. L., Bach, K. & Marioni, J. C. Pooling across cells to normalize single-cell RNA sequencing data with many zero counts. Genome Biol. 17, 75 (2016).
73. Wolf, F. A., Angerer, P. & Theis, F. J. SCANPY: large-scale single-cell gene expression data analysis. Genome Biol. 19, 15 (2018).
74. Haghverdi, L., Buettner, F. & Theis, F. J. Diffusion maps for high-dimensional single-cell analysis of differentiation data. Bioinformatics 31, 2989-2998 (2015).
75. Traag, V. A., Waltman, L. & van Eck, N. J. From Louvain to Leiden: guaranteeing well-connected communities. Sci. Rep. 9, 1-12 (2019).
76. Tan, Y. & Cahan, P. SingleCellNet: A Computational Tool to Classify Single Cell RNA-Seg Data Across Platforms and Across Species. Cell Systems 9, 207-213.e2 (2019).
77. Aibar, S. et al. SCENIC: single-cell regulatory network inference and clustering. Nat. Methods 14, 1083-1086 (2017).
78. Van de Sande, B. et al. A scalable SCENIC workflow for single-cell gene regulatory network analysis. Nat. Protoc. 15, 2247-2276 (2020).
79. Lambert, S. A. et al. The Human Transcription Factors. Cell 172, 650-665 (2018).
80. Finak, G. et al. MAST: a flexible statistical framework for assessing transcriptional changes and characterizing heterogeneity in single-cell RNA sequencing data. Genome Biol. 16, 278 (2015).
81. Lavallee, V.-P. et al. EVI1-rearranged acute myeloid leukemias are characterized by distinct molecular alterations. Blood 125, 140-143 (2015).
82. Lavallee, V.-P. et al. The transcriptomic landscape and directed chemical interrogation of MLL-rearranged acute myeloid leukemias. Nat. Genet. 47, 1030-1037 (2015).
83. Lavallee, V.-P. et al. Identification of MYC mutations in acute myeloid leukemias with NUP98-NSD1 translocations. Leukemia 30, 1621-1624 (2016).
84. Lavallee, V.-P. et al. RNA-sequencing analysis of core binding factor AML identifies recurrent ZBTB7A mutations and defines RUNX1-CBFA2T3 fusion signature. Blood 127, 2498-2501 (2016).
85. Lavallee, V.-P. et al. Chemo-genomic interrogation of CEBPA mutated AML reveals recurrent CSF3R mutations and subgroup sensitivity to JAK inhibitors. Blood 127, 3054-3061 (2016).
86. Maiga, A. et al. Transcriptome analysis of G protein-coupled receptors in distinct genetic subgroups of acute myeloid leukemia: identification of potential disease-specific targets. Blood Cancer J. 6, e431 (2016).
87. Simon, L. et al. Chemogenomic Landscape of RUNX1-mutated AML Reveals Importance of RUNX1 Allele Dosage in Genetics and Glucocorticoid Sensitivity. Clin. Cancer Res. 23, 6969-6981 (2017).
88. Baccelli, I. et al. A novel approach for the identification of efficient combination therapies in primary human acute myeloid leukemia specimens. Blood Cancer J. 7, e529 (2017).
89. Lavallee. V.-P. et al. Transcriptomic landscape of acute promyelocytic leukemia reveals aberrant surface expression of the platelet aggregation agonist Podoplanin. Leukemia 32, 1349-1357 (2018).
90. Moison, C. et al. Complex karyotype AML displays G2/M signature and hypersensitivity to PLK1 inhibition. Blood Adv 3, 552-563 (2019).
91. Baccelli. I. et al. Mubritinib Targets the Electron Transport Chain Complex I and Reveals the Landscape of OXPHOS Dependency in Acute Myeloid Leukemia. Cancer Cell 36, 84-99.e8 (2019).
92. Bisaillon, R. et al. Genetic characterization of ABT-199 sensitivity in human AML. Leukemia 34, 63-74 (2020).
93. Deshwar, A. G. et al. PhyloWGS: reconstructing subclonal composition and evolution from whole-genome sequencing of tumors. Genome Biol. 16, 35 (2015).
94. Miller, C. A. et al. Visualizing tumor evolution with the fishplot package for R. BMC Genomics 17, 880 (2016).
95. Xu, C. et al. Probabilistic harmonization and annotation of single-cell transcriptomics data with deep generative models. Mol. Syst. Biol. 17, e9620 (2021).
96. Lotfollahi, M. et al. Query to reference single-cell integration with transfer learning. bioRxiv 2020.07.16.205997 (2020) doi: 10.1101/2020.07.16.205997.
97. Smirnov, P. et al. Evaluation of statistical approaches for association testing in noisy drug screening data. arXiv [stat.AP] (2021).
98. Galkin, O. et al. SIRPaFc treatment targets human acute myeloid leukemia stem cells. Haematologica 106, 279-283 (2021).

1. A method of predicting treatment response to a drug in a patient with leukemia, wherein the drug had been predetermined to preferentially target either primitive or mature leukemic cells, the method comprising:
(a) determining the expression level of at least 3 genes in a test sample from the subject selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, and GPR56;
(b) calculating a primitiveness score comprising the weighted sum expression of each of the at least 3 genes; and either
(c) predicting that the patient will be sensitive to treatment by the drug if (i) the drug preferentially targets primitive leukemic cells and the calculated primitiveness score is high in reference to a control cohort of leukemia patients; or (ii) the drug preferentially targets mature leukemic cells and the calculated primitiveness score is low in reference to a control cohort of leukemia patients; or
(d) predicting that the patient will be resistant to treatment by the drug if (i) the drug preferentially targets primitive leukemic cells and the calculated primitiveness score is low in reference to a control cohort of leukemia patients; or (ii) the drug preferentially targets mature leukemic cells and the calculated primitiveness score is high in reference to a control cohort of leukemia patients.

2. The method of claim 1, wherein the calculated primitiveness score is high if it is higher than the median score of the control cohort of leukemia patients and the calculated primitiveness score is low if it is lower than the median score of the control cohort of leukemia patients.

3. The method of claim 1, wherein the leukemia is acute myeloid leukemia.

4. The method of claim 1, wherein the at least 3 genes consists of DNMT3B, LAPTM4B, CDK6, CPXM1, NGFRAP1, CD34, and GPR56.

5. The method of claim 1, further comprising treating the patient with the drug if the patient has been predicted to be sensitive to treatment by the drug according to the patient's primitiveness score.

6. The method of claim 1, wherein the drug preferentially targeting primitive leukemia cells is Selinexor, Venetoclax, Erlotinib, GSK-1838705A, Gefitinib, Canertinib (CI-1033), Pelitinib (EKB-569), PHA-665752, Barasertib (AZD1152-HQPA), Palbociclib, Sorafenib, NVP-ADW742, NF-kB Activation Inhibitor, Bay 11-7085, Lenalidomide, Afatinib (BIBW-2992), SR9011, KU-55933, KW-2449, Roscovitine (CYC-202), LY-333531, NVP-TAE684, Vandetanib (ZD6474), Pazopanib (GW786034), Vargetef, Dovitinib (CHIR-258), Vatalanib (PTK787), Vemurafenib (PLX-4032), Tipifarnib, PLX-4032, BAY 11-7082, Lomustine, MLN8237, PLX-4720, Azacitidine, 5-lodotubercidin, NVP-TAE-684, Mubritinib, Decitabine, BI-2536, NVP-LDE-225, Tosedostat, SB 218078, Flavopiridol, Melphalan, AS101, BSI-201, ABT-263, Fenretinide, ARQ-197, Tozasertib, BAY 11-7085, PD0332991, Topotecan HCl, Pravastatin, Etoposide, Vinblastine sulfate, GDC-0449, Pemetrexed, TG-101348, PIK-75, Acrichine, AS-605240, Gemcitabine HCl, ABT-888, XL-147, Bexarotene, Crizotinib, Erlotinib HCl, Etodolac, Otava 7015980251, BIBW 2992, FTI-276, Irinotecan HCl, BML 277, Vincristine sulfate, Arsenic trioxide, PF-04449913, GF109203X, or CC-90009.

7. The method of claim 1, wherein the drug preferentially targeting mature leukemic cells is GW-2580, JNJ-28312141, INK-128, Staurosporine, Idelalisib, MK-2206, PRT062607, Cediranib, Linifanib, Go6976, DasaUnib, PLX-4720, CI-1040, 17-AAG, Tandutinib, Rapamycin, PKI-587, Everolimus, Temsirolimus, Panobinostat, Selumetinib (AZD6244), GDC-0941, Nilotinib, BEZ235, MK-2206, SNS-032 (BMS-387032), Flavopiridol, TG100-115, Trametinib (GSK1120212), Cediranib (AZD2171), Bortezomib (Velcade), or Fedratinib.

8. A composition comprising a plurality of isolated nucleic acid sequences, wherein each isolated nucleic acid sequence hybridizes to:
(a) the mRNA of at least 3 genes selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, GPR56; and/or
(b) a nucleic acid complementary to a),
wherein the composition is used to measure the level of expression of at least 3 genes.

9. The composition of claim 8, wherein the at least 3 genes consists of DNMT3B, LAPTM4B, CDK6, CPXM1, NGFRAP1, CD34, and GPR56.

10. An array comprising, for each of at least 3 genes selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, GPR56, one or more polynucleotide probes complementary and hybridizable thereto.

11. The array of claim 10, wherein the at least 3 genes consists of DNMT3B, LAPTM4B, CDK6, CPXM1, NGFRAP1, CD34, and GPR56

12. A computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the method of claim 1.

13. A computer implemented product for predicting treatment response to a drug in a patient with leukemia, wherein the drug had been predetermined to preferentially target either primitive or mature leukemic cells, the computer implemented product comprising:
(a) a means for receiving values corresponding to a subject expression profile comprising at least 3 genes from the subject selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, and GPR56;
(b) a database comprising a reference expression profile representing a control, wherein the subject expression profile and the reference profile each have at least one value representing the expression level of at least 3 genes selected from the group consisting of DNMT3B, ZBTB46, NYNRIN, ARHGAP22, LAPTM4B, MMRN1, DPYSL3, KIAA0125, CDK6, CPXM1, SOCS2, SMIM24, EMP1, NGFRAP1, CD34, AKR1C3, GPR56;
(c) a means for calculating a primitiveness score comprising the weighted sum expression of each of the at least 3 genes; and either
(d) a means for outputting a prediction that that the patient will be sensitive to treatment by the drug if (i) the drug preferentially targets primitive leukemic cells and the calculated primitiveness score is high in reference to a control cohort of leukemia patients; or (ii) the drug preferentially targets mature leukemic cells and the calculated primitiveness score is low in reference to a control cohort of leukemia patients; or
(e) a means for outputting a prediction that the patient will be resistant to treatment by the drug if (i) the drug preferentially targets primitive leukemic cells and the calculated primitiveness score is low in reference to a control cohort of leukemia patients; or (ii) the drug preferentially targets mature leukemic cells and the calculated primitiveness score is high in reference to a control cohort of leukemia patients.

14. A method for selecting a drug in a patient with leukemia, wherein the drug had been predetermined to preferentially target either primitive or mature leukemic cells, the method comprising the method of claim 1, and further comprising selecting the drug if the patient had been predicted to be sensitive to treatment by the drug according to the patient's primitiveness score.

15. (canceled)

16. (canceled)
